(19) United States
(12) Patent Application Publication
(10) Pub. No.: US 2023/0265377 A1
(43) Pub. Date: Aug. 24, 2023

(54) REFERENCE CANCELING SYSTEMS, DEVICES, AND METHODS FOR DETERMINING TISSUE CHARACTERISTICS IN VITRO

(71) Applicant: University of Washington, Seattle, WA (US)

(72) Inventors: Nathan J. Sniadecki, Seattle, WA (US); Ty Higashi, Seattle, WA (US); Daniel Moskowitz, Seattle, WA (US); Jevne Micheau-Cunningham, Seattle, WA (US); Robert Bruce Darling, Seattle, WA (US)

(73) Assignee: University of Washington, Seattle, WA (US)

(21) Appl. No.: 17/766,876

(22) PCT Filed: Oct. 7, 2020
(86) PCT No.: PCT/US2020/054587

§ 371 Date: Apr. 6, 2022

Related U.S. Application Data
(60) Provisional application No. 62/913,116, filed on Oct. 9, 2019.

(51) Int. Cl. C12M 1/42; G01N 33/483; C12M 1/32; C12M 3/00

(52) U.S. Cl. CPC C12M 35/04; G01N 33/4833; C12M 23/12; C12M 21/08

(57) ABSTRACT

Devices and methods configured to determine characteristics of tissue specimens are provided. Representative tissue analysis devices include a sensing module and a reference module. The sensing module includes a first post and a second post configured to have the tissue specimen affixed thereto, and a displacement sensor configured to output a displacement signal corresponding to a displacement of the first post. The reference module includes a reference sensor configured to output a reference signal corresponding to a reference input such as an ambient magnetic field. The devices further include instructions that determine: a displacement value based upon the displacement signal; a reference value based upon the reference signal; a reference-canceled displacement value based upon the displacement value and the reference value; and a characteristic of the tissue specimen based upon the reference-canceled displacement value.

### CROSS-REFERENCE TO RELATED APPLICATION

This application claims the benefit of U.S. Provisional Application Ser. No. 62/913,116, filed Oct. 9, 2019, the entirety of which is hereby incorporated by reference for all purposes.

### STATEMENT OF GOVERNMENT LICENSE RIGHTS

This invention was made with government support under Grant No. 1661730, awarded by the National Science Foundation. The government has certain rights in the invention.

### BACKGROUND

It is desirable to measure the baseline strength of tissues under contraction—for example, cardiomyocytes, but also other tissues—as well as those same tissues following an applied treatment with known, unknown, and/or expected effects on the tissue regarding its strength of contraction, as well as other characteristics related to the function of contraction, including, but not limited to: the absolute steady-state force, relative force, strain, frequency, the sustainable duration of contraction for a given stimulus, the time response of the tissue for a given stimulus, etc. Such measurements may allow doctors and researchers to assess the maturity and viability of the tissue, and thereby enhance the likelihood of successful repair.

### SUMMARY

The present disclosure relates to devices and methods for culturing and analysis of biological tissue and/or cells. Such tissue and cells include, but are not limited to, muscle tissue (e.g., engineered heart tissue, smooth muscle tissue, and skeletal muscle tissue) and non-muscle tissue (e.g., ligament tissue and suture tissue).

In an aspect, the present disclosure provides tissue analysis devices for determining a characteristic (e.g., force, strain) of at least one biological tissue specimen. The tissue analysis devices include a sensing module, a reference module. The sensing module includes a first post disposed on a base, having a magnetic material disposed therein, and configured to have the tissue specimen attached thereto; a second post disposed on the base and configured to have the tissue specimen attached thereto; and a displacement sensor configured to output a displacement signal corresponding to a displacement of the first post. The reference module includes a reference sensor configured to output a reference signal corresponding to a reference input. In addition, the tissue analysis devices include a non-transitory machine readable storage medium storing logic, which when executed by a processor, causes the processor to perform operations, including: determining a displacement value based upon the displacement signal; determining a reference value based upon the reference signal; determining a reference-canceled displacement value based upon the displacement value and the reference value; and determining the characteristic based upon the reference-canceled displacement value.

In any of the embodiments disclosed herein, the sensing module further includes a third post, wherein the first post, the second post, and the third post are arranged in a triangular configuration. In any of the embodiments disclosed herein, the sensing module further includes a third post and a fourth post, and wherein the first post, the second post, the third post, and the fourth post are arranged in a rectangular configuration. In any of the embodiments disclosed herein, the sensing module further includes a third post, a fourth post, and a fifth post, wherein the first post, second post, third post, fourth post, and fifth post are arranged in a pentagonal configuration.

In any of the embodiments disclosed herein, determining the reference-canceled displacement value is based upon subtracting the reference value from the displacement value.

In any of the embodiments disclosed herein, determining the displacement value includes multiplying the displacement signal by a linear factor and by a non-linear factor.

In any of the embodiments disclosed herein, determining the displacement value does not include frequency filtering the displacement signal before multiplying the displacement signal by the linear factor.

In any of the embodiments disclosed herein, determining the reference value includes multiplying the reference signal by a linear factor.

In any of the embodiments disclosed herein, determining the characteristic includes multiplying the reference-canceled displacement value by a linear factor.

In any of the embodiments disclosed herein, the linear factor is a correlation factor between the displacement of the first post and a force exerted by the tissue specimen.

In any of the embodiments disclosed herein, determining the displacement value includes multiplying the displacement signal by a first linear factor and by a non-linear factor, determining the reference value includes multiplying the reference signal by a second linear factor, different from the first linear factor, and determining the characteristic includes multiplying the reference-canceled displacement value by a third linear factor, different from the first linear factor and the second linear factor.

In any of the embodiments disclosed herein, the displacement sensor is disposed a first distance away from the first post and the reference sensor is disposed a greater second distance away from the first post. In any of the embodiments disclosed herein, the second distance is sufficiently large that the reference sensor does not sense any signal amplitude from the tissue specimen.

In any of the embodiments disclosed herein, the sensing module further includes: a third post configured to be adhered to a second tissue specimen and having a second magnetic material disposed therein; a fourth post configured to be adhered to the second tissue specimen; and a second displacement sensor configured to output a second displacement signal corresponding to a displacement of the third post. In any of the embodiments disclosed herein, the reference sensor is disposed equidistant from the displacement sensor and the second displacement sensor.

In any of the embodiments disclosed herein, the first post and the second post are disposed in a well of a culture dish, and the displacement sensor is disposed directly beneath the first post on a printed circuit board.

In any of the embodiments disclosed herein, the displacement signal corresponds to a change in a local magnetic field caused by the displacement of the first post, and the reference signal corresponds to an ambient magnetic field.

In any of the embodiments disclosed herein, the displacement sensor and the reference sensor have a common orientation.

In any of the embodiments disclosed herein, the displacement sensor and the reference sensor are a same sensor type selected from the group consisting of: a Giant Magnetoresistive (GMR) sensor, a flux gate, a Hall sensor, and an anisotropic magnetic resistance magnetometer.

In any of the embodiments disclosed herein, the characteristic is an absolute force.

In any of the embodiments disclosed herein, the displacement value includes a plurality of displacement value components corresponding to sensed magnetic fields in a plurality of axes; the reference value includes a plurality of reference value components corresponding to reference magnetic fields in the plurality of axes; and determining the reference-canceled displacement value is based upon subtracting at least one of the plurality of displacement value components from a corresponding one of the plurality of reference value components.

In another aspect, the present disclosure provides methods, e.g., methods of determining a characteristic of a tissue specimen. The methods include affixing a tissue specimen to a first post and a second post; sensing a displacement of the first post relative to the second post; sensing a reference input while sensing the displacement of the first post relative to the second post; outputting a displacement signal based upon the displacement of the first post relative to the second post; outputting a reference signal based upon the reference input; determining a displacement value based upon the displacement signal; determining a reference value based upon the reference signal; determining a reference-canceled displacement value based upon the reference value and the displacement value; and determining a characteristic of the tissue specimen based upon the reference-canceled displacement value.

In any of the embodiments disclosed herein, determining the displacement value includes multiplying the displacement signal by a linear factor and by a non-linear factor.

In any of the embodiments disclosed herein, determining the displacement value does not include frequency filtering the displacement signal before multiplying the displacement signal by the linear factor.

In any of the embodiments disclosed herein, determining the reference value includes multiplying the reference signal by a linear factor.

In any of the embodiments disclosed herein, determining the characteristic includes multiplying the reference-canceled displacement value by a linear factor.

In any of the embodiments disclosed herein, the linear factor is a correlation between displacement of the first post and the characteristic of the tissue specimen.

In any of the embodiments disclosed herein, determining the displacement value includes multiplying the displacement signal by a first linear factor and by a non-linear factor, determining the reference value includes multiplying the reference signal by a second linear factor, and determining the characteristic includes multiplying the reference-canceled displacement value by a third linear factor.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows a side view of a tissue analysis device configured to magnetically detect characteristics of a tissue specimen.

FIG. 2 shows a circuit for detecting a change in a magnetic field associated with a displacement of a tissue specimen.

FIG. 3 shows the example device of FIG. 1 including external magnets used to simulate preload and afterload of tissue specimens.

FIG. 4A shows a graph of preload and afterload forces varied over time with an external magnet.

FIG. 4B shows a length-force graph of a tissue specimen.

FIG. 4C shows a graph exhibiting cardiac pressure-volume (PV) loops representing changes in pressure during preload and/or afterload in cardiac tissue.

FIG. 5 shows a graph showing varying post position and resulting voltage resulting from the beating of cardiac tissue in the various devices described in the present disclosure.

FIG. 6 shows a representative method for magnetically determining force exerted by a tissue specimen.

FIG. 7 shows an example computing device configured to perform the various methods described herein.

FIG. 8 shows a reference canceling tissue analysis device, in accordance with an embodiment of the present disclosure.

FIGS. 9A-9E show different configurations of an aspect of tissue analysis devices of the present disclosure.

FIG. 10 shows one representative configuration of another aspect of tissue analysis devices of the present disclosure.

FIG. 11 shows a representative circuit for a tissue analysis device, in accordance with the present disclosure.

FIG. 12 shows a representative layout of a printed circuit board of a tissue analysis device, in accordance with the present disclosure.

FIGS. 13A and 13B show aspects of a tissue analysis device, in accordance with the present disclosure.

FIG. 14 shows a method for determining a characteristic of a tissue specimen, in accordance with the present disclosure.

### DETAILED DESCRIPTION

In the following description, reference is made to the accompanying drawings that illustrate several embodiments of the present disclosure. It is to be understood that other embodiments may be utilized and system or process changes may be made without departing from the spirit and scope of the present disclosure. The following detailed description is not to be taken in a limiting sense, and the scope of the embodiments of the present invention is defined only by the claims.

Various embodiments of the present disclosure provide improved systems and methods for magnetic detection and determination of forces and other characteristics (e.g., strain and stress) of biological tissue specimens (hereinafter referred to as “tissue specimens”), including muscle tissue and non-muscle tissue specimens. Representative muscle tissues include cardiac cells (e.g., cardiomyocytes), skeletal muscle, smooth muscle, and the like. Representative non-muscle tissue includes ligament tissue, suture tissue, and the like. To facilitate understanding, the present disclosure occasionally describes the devices and methods in the context of cardiomyocytes; however, the skilled artisan will appreciate that the devices and methods described herein are not limited to cardiomyocytes, but are applicable to at least the other tissues described above.

Embodiments of the present disclosure improve a signal to noise ratio for determination and analysis of characteristics of a tissue specimen. Additionally, these embodiments reduce data storage requirements for tissue characteristic determination, e.g., relative to optical methods of analyzing tissue specimens. The techniques described herein allow for repeatable, accurate, and precise results. As used herein, “cardiac tissue” and/or “myocardial tissue” may refer to single cardiomyocytes and/or multiple cardiomyocytes fused to form a tissue. In the following detailed description these terms may sometimes be used interchangeably.

Embodiments of the present disclosure are configured such that tissue specimens may be cultured and affixed to polymeric microposts, referred to herein as “posts.” Analyzing characteristics of the tissue specimens (e.g., force response) can provide information about the effects of a variety of reagents and treatments to which the tissue specimens are exposed as well as information about the development and maturity of the tissue specimens. In some embodiments, small tissues made up of cardiomyocytes may be cultured and affixed between microposts (e.g., with an adhesive). The tissue specimens may be seeded within a fiber matrix. In some embodiments, the fiber matrix may comprise collagen, fibrin, matrigel, silicone, and/or other polymeric substances. At least some of the microposts to which the cardiac tissue is affixed may be designed in such a way as to be flexible. In various embodiments, the diameter, length, or material of the microposts may be selected in order to impart a desired flexibility or rigidity to the microposts. The cardiac tissue may be effective to bend the flexible microposts upon contraction of the cardiac tissue.

In this disclosure, microposts may sometimes be referred to as “posts.” Additionally, in some cases, the posts described herein may have a wide variety of dimensions. As such, the prefix “micro” used in conjunction with the term “posts” does not describe all possible and intended posts contemplated by the present disclosure. As described in further detail below, the force generated by the tissue specimen may be determined based on the amount by which the post is displaced (e.g., bent or deflected).

Previous attempts at seeding tissue specimens and determining characteristics from such tissue specimens (e.g., engineered heart tissues) have encountered a variety of obstacles. For example, sophisticated image analysis may be used to monitor the motion of the microposts to which the cardiac tissue is affixed. The force exerted by the cardiac tissue may be determined based on the motion; however, a dedicated microscope and highly complex computer vision techniques are required to monitor the force of the cardiac tissue over time. The computer processing requirements and data storage requirements for processing and storing the optical data is relatively high, especially as compared with the techniques described herein. Additionally, expansion beyond a single 24-well plate requires additional microscopes and/or a significant set-up and tear-down time for each point of force measurement. As such, massively parallel studies using such techniques may be prohibitively expensive and/or time consuming. Some other methods of monitoring the force development of engineered cardiomyocyte tissues require destructive methods or merely monitor the electrophysiology and not the actual force generation of the cardiac tissue.

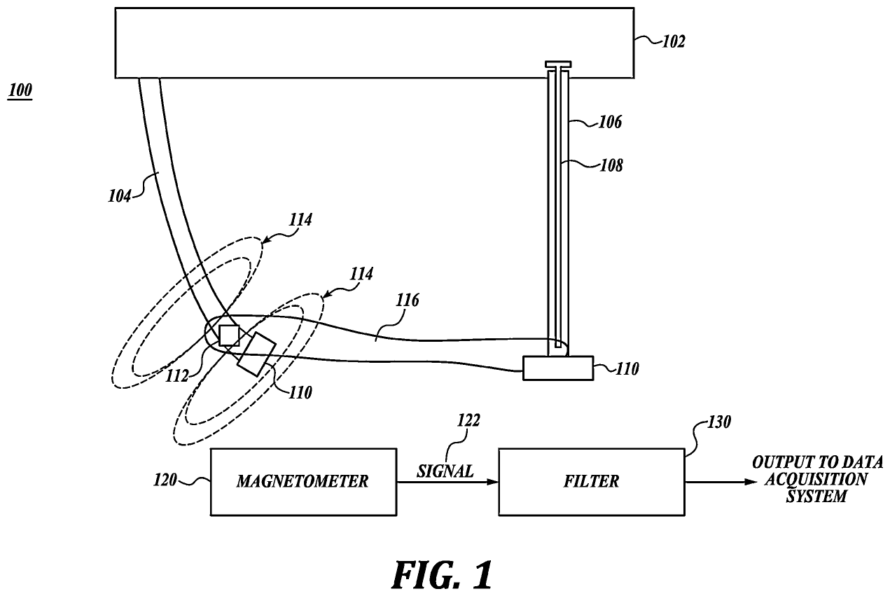

FIG. 1 depicts a side view of a tissue analysis device 100 that can be used to magnetically detect tissue forces and other characteristics, in accordance with various embodiments of the present disclosure. Device 100 may include a base 102 and a plurality of posts including, for example, post 104 and post 106. In some embodiments, posts 104 and/or 106 may comprise a polydimethylsiloxane (PDMS) polymer. In various embodiments, the PDMS polymer may be formed from a four-part acrylic mold or another mold. In various embodiments, base 102 may be comprised of a biocompatible matrix material such as collagen, fibrin, Matrigel, and/or any other suitable material to which to couple and/or affix microposts. Although two posts 104 and 106 are depicted in FIG. 1, any number of posts may be used in accordance with various embodiments of the present disclosure. For example, some embodiments include a third post, and wherein the first post, the second post, and the third post are arranged in a triangular configuration. Some embodiments include a third post and a fourth post, wherein the first post, the second post, the third post, and the fourth post are arranged in a rectangular configuration. Some embodiments include a third post, a fourth post, and a fifth post, wherein the first post, the second post, the third post, the fourth post, and the fifth post arranged in a pentagonal configuration. Such embodiments are representative, not limiting.

In some embodiments, there is a gap of between 4 mm and 20 mm between adjacent posts, such as posts 104 and 106. In some further embodiments, posts 104 and 106 may be between approximately 5 mm and 24 mm tall and between 0.5 mm and 3 mm in diameter.

In other embodiments, posts may be taller or shorter and may have larger or smaller diameters relative to the ranges previously mentioned, according to the desired implementation. Similarly, the gaps between posts may in some cases be smaller than 4 millimeters, depending on the desired implementation. As is described in further detail below, the dimensions of the posts may be selected in order to impart rigidity and/or flexibility to the posts. In some embodiments, posts arranged in an array may be spaced for insertion into a multi-well plate such as, for example, a 24-well plate. In various embodiments, each pair of posts, such as posts 104 and 106, in an array of posts may be appropriately spaced and located such that each pair of posts may correspond with and fit into a well of a multi-well plate.

The base 102 may be rigid or flexible and may be designed to interface with a 24-well or other-numbered well plate so that the tips 110 of pairs of posts (e.g., posts 104 and 106) of base 102 may be inverted to fit into individual wells of the plate. Tissue specimen 116 may be cultured and may affixed to (e.g., with an adhesive) tips 110 of the posts 104, 106 such that the cardiac tissue grows “between” the two posts. For example, tissue specimen 116 depicted in FIG. 1 may adhere to posts 104 and 106 and may be grown between the two posts. In some embodiments, tissue specimen 116 may be cultured in the individual wells. In various other embodiments, and as described in further detail below, wells of a multi-well plate into which tips 110 of posts 104, 106 are inserted may include solutions comprising nutrients and/or therapeutic agents. In some embodiments, cardiac tissue adhered to posts 104, 106 may be exposed to therapeutic agents in order to test the efficacy of the therapeutic agents by measuring the response of the cardiac tissue to the agents.

A first proximal end of posts 104 and 106 may be coupled to base 102 and a second, distal end of posts 104 and 106 may comprise a tip 110. While in some embodiments the tip 110 may be non-uniform relative to the remainder of the posts in order to promote tissue attachment, in various other embodiments, the tip 110 may be relatively uniform with respect to the post, depending on the desired implementation. In various embodiments, the posts may comprise a polymeric material and may be less than about 2 millimeters in diameter. Post 104 may be sufficiently flexible such that the tip 110 of post 104 may be deflected away from an at-rest position in response to the contraction of cardiac tissue affixed to post 104. For example, tissue specimen 116 may be affixed to post 104 at or near tip 110 of post 104. Tissue specimen 116 may also be affixed to post 106, or another post and/or object. When tissue specimen 116 contracts due to the spontaneous beating of cardiac tissue, the force of the contraction causes post 104 to bend or deflect from an at-rest position to a second, deflected position. In FIG. 1, post 104 is shown in a deflected position, bent away from a vertical rest position.

Post 106 may include a rigid insert 108 to impart rigidity to post 106 in order to prevent and/or limit the deflection or other movement of post 106 from the vertical rest position in response to the contraction of tissue specimen 116. Rigid insert 108 may comprise silicon glass, metal, plastic and/or any other material of sufficient rigidity to prevent and/or reduce the deflection of post 106 during the contraction of tissue specimen 116.

In some other embodiments, post 106 may not include a rigid insert and may accordingly exhibit similar flexibility as other posts affixed to base 102. In still other embodiments, the diameter and/or length of posts may be altered in order to impart the desired level of flexibility to various posts. For example, if it is desired that some posts be more flexible and other posts be less flexible, some posts may be formed with a smaller diameter and/or a greater length in order to impart greater flexibility along the length of the post. Similarly, other posts may be formed with a larger diameter and/or a shorter length in order to impart rigidity/limit flexibility. Additionally, although the description herein refers to posts of a cylindrical shape, posts may instead be formed with other shapes. For example, the posts may be formed in a parallelepiped shape or other polygonal shape.

As depicted in FIG. 1, post 104 may comprise a magnetic material 112 such as neodymium and/or another magnet embedded within or otherwise coupled to the post. In some embodiments, tip 110 of post 104 may be configured to prevent the post 104 from tearing due to the embedding of magnetic material 112. In various further embodiments the magnetic material may be smaller than 2 mm³. In various embodiments, the magnetic material 112 may be disposed at or near the tip of post 104 such that when post 104 is deflected the magnet is accordingly displaced from a first position to a second position by the deflection of the post 104.

Magnetic material 112 produces a magnetic field 114. Deflection of post 104 may cause magnetic material 112 to be displaced and/or to rotate relative to the original position of magnetic material 112. Accordingly, the magnetic field 114 associated with magnetic material 112 may similarly translate and/or rotate due to the translation and/or rotation of magnetic material 112.

A displacement sensor 120 (e.g., a magnetometer) disposed proximate to post 104 may detect the changes in the strength of a detected magnetic field as the magnetic material 112 and magnetic fields 114 move closer to and away from the displacement sensor 120 with the beating of the tissue specimen 116. The displacement sensor 120 may be disposed at various positions relative to positions of post 104 and post 106. Displacement sensor 120 may be positioned such that displacement sensor 120 can detect a change in the magnetic field due to deflection of magnetic material 112 from a first position to a second position. In various embodiments, displacement sensor 120 may be positioned within 0.1 mm-10 mm from post 104. In some other embodiments, displacement sensor 120 may be positioned between 11-30 millimeters from post 104. In various other embodiments, displacement sensor 120 may be positioned closer to or further from post 104, depending on the type of magnetometer and/or the type of magnetic material used.

In some embodiments, the change in magnetic field strength detected by the displacement sensor 120 may be on the order of microteslas and may affect only a local region within the well in which posts 104 and 106 are disposed. Accordingly, magnetic field changes associated with the beating of tissue specimen 116 may be distinguished from magnetic field changes associated with beating of cardiac tissue adhered to other posts, and/or disposed in other wells of a multi-well plate.

In various embodiments, the displacement sensor 120 is a giant magnetoresistive (GMR) sensor, a flux gate, a Hall sensor, anisotropic magnetic resistance magnetometers, or similar magnetometer. Embodiments are generally described herein in the context of GMR sensors; however, this is representative and not limiting. In some embodiments, an array of displacement sensors 120 may be arranged such that a single magnetometer may be associated with each well of a multi-well plate. Accordingly, the changes in magnetic field strength associated with the cardiac tissue disposed in each well of a multi-well plate may be detected and distinguished from other wells. The change in field strength results in an output displacement signal 122. The displacement signal 122 may comprise a voltage output from displacement sensor 120.

In various embodiments, magnetometers and/or arrays of magnetometers may be arranged on printed circuit boards along with other circuitry for filtering, amplifying, and/or reference canceling displacement signal 122. In some embodiments where displacement sensors 120 are GMR-based magnetic sensors, the displacement sensors 120 may include resistances arranged in a Wheatstone bridge configuration that results in a decrease in voltage of displacement signal 122 when the magnetic field detected by the GMR sensor increases. Each measurement from the Wheatstone bridge of the GMR sensor may be output as a displacement signal 122 corresponding to a displacement of the post 104 relative to the GMR sensor. The displacement signal can be a voltage, current, digital signal, or other signal. The displacement signal is described herein as a voltage signal; however, this is representative and not limiting.

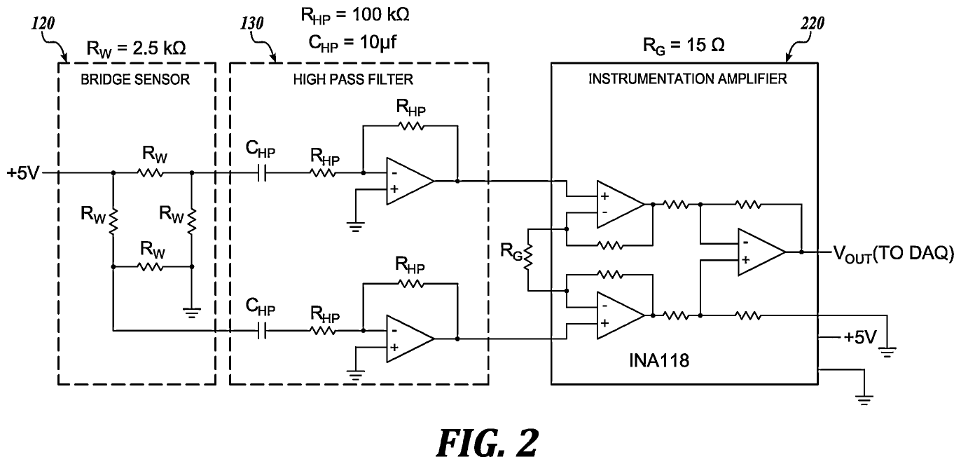

In some embodiments, the displacement signal 122 is output to a high pass filter, a bandpass filter, and/or reference canceling circuit, and amplified using one or more operational and/or instrumentation amplifiers. See, for example, FIG. 2 depicting an example of circuitry including displacement sensor 120, filter 130 (a high pass filter in FIG. 2), and an instrumentation amplifier 220. Some embodiments described herein, e.g., some reference canceling embodiments, do not include such frequency filtration.

The output voltage from filter 130 may be detected by a data acquisition system and may be used to determine a force associated with the beating of the tissue specimen 116, as discussed in further detail below. Although a high pass filter is depicted in the example circuitry shown in FIG. 2, in some other embodiments, low pass and/or bandpass filters may be used, in accordance with the various embodiments described herein, depending on the desired frequencies to be captured and passed to the data acquisition system. In still other embodiments described herein, a reference canceling circuit is utilized instead of low pass, bandpass, and/or high pass filters in order to preserve all frequencies represented in the displacement signal 122. In still other embodiments, a reference canceling circuit is utilized in addition to one or more low pass, bandpass, and/or high pass filters.

Displacement sensors 120 may be soldered to printed circuit boards containing conditioning circuitry. In some embodiments, the displacement sensors 120 may comprise Wheatstone bridge configurations and may be routed to high-pass filters to reduce long-term drift of the system. Filters, such as the high pass filter 130 depicted in FIG. 2, may be designed to have a cut-off frequency that passes cardiac tissue contraction frequencies while excluding low-frequency ambient noise. In some embodiments, high pass filters 130 may have a cut-off frequency of approximately 0.01-0.3 Hz. In various other embodiments, high pass filters 130 may have a cut-off frequency of approximately 0.1-0.25 Hz. In still other embodiments high pass filters 130 may have a cut-off frequency of approximately 0.16 Hz-0.5 Hz. Although, various frequency ranges are provided for illustrative purposes, other ranges of cut-off frequencies may be used in accordance with the present disclosure. Identification of the frequency of rhythmic beating of the cardiac tissue can, in some embodiments, serve as an upper limit for the cutoff frequency of the high pass filters, although often a lower cut-off frequency may be used in order to avoid data loss from slower than average beating of cardiac tissue. Additionally, band pass and/or low pass filters may be used in various implementations in order to filter out frequencies lower than and/or higher than the frequencies associated with the beating of the cardiac tissue. Filter 130 may be effective to offset drift in the detected signals due to temperature fluctuations and/or due to ambient magnetic fields of the environment in which device 100 is situated. Embodiments of the present disclosure, for example some reference canceling embodiments described below, exclude such low-pass, high-pass, and/or band-pass filters, for the advantage of preserving the frequencies correlative to characteristics of the tissue specimen.

Signals from filters 130 may be routed through instrumentation amplifiers such as instrumentation amplifier 220 depicted in FIG. 2 before going through a data acquisition system. The data acquisition system may be effective to monitor and record the frequencies of cardiac tissue contraction and force. In addition, the data acquisition system may record the timing of additions of fluids, such as therapeutic agents and/or nutrients. The data acquisition system may include one or more processing elements and/or one or more memories effective to store data received from filter 130 depicted in FIG. 1.

Embodiments of the present disclosure may utilize some or all of the circuitry described above, i.e., to form additional embodiments.

### Magnetic Model

The embodiments below describe experimental methods used to validate a system for magnetic detection of myocardial forces. Although particular data and instrumentation are described in the discussion below, other instruments (e.g., magnets, filters, materials) may be used in accordance with the present disclosure and such other instruments may yield different values than those discussed below for purposes of example.

A model of the system was developed in Matlab (although any suitable programming language may be used) by treating the embedded magnetic material 112 as a point dipole. A point dipole may be a fair approximation for the magnetic material 112, as the distance between the magnetic material 112 and the displacement sensor 120 was much greater than the size of the magnetic material 112. In some embodiments, the dipole strength of 1 mm3 neodymium magnets may have a dipole strength of M=7.5eₓ+1.5e_{γ}+1.5e^{z} mAm². The x-component of the stray field was determined for an array on the plane of the sensor to determine the effect of adjacent posts as well as the optimal location for the sensor. The projection was determined based on the magnetic field of a point dipole: where m is the magnetic moment described above, B is the magnetic field change, and r is the distance from the current location of the magnet to the position in the plane of the sensor. In some embodiments, the sensor is at least 10 mm away from the magnet in the vertical direction. In various embodiments, an optimum position of the displacement sensor 120 relative to the post 104 may comprise locating displacement sensor 120 a 1-5 mm ahead of the post 104.

The magnetic field change B can be used to determine the force of the tissues after a calibration is performed on the system. The force exerted by the cardiac tissue on the flexible post 104 is proportional to the distance the tip 110 of the flexible post 104 moves, determined by the stiffness and dimensions of the flexible post 104. The distance the tip 110 of the flexible post 104 moves causes a change in Bat the displacement sensor 120, which in turn creates a difference in the displacement signal (i.e., voltage) read at a computing device. A calibration can be performed by manually moving the tips of the posts a specified distance (thus generating a known force) and monitoring the corresponding voltage change in the system. The voltage change is due to the B field change.

Displacement sensors 120 may be spaced in an array such that adjacent displacement sensors 120 do not have measurable signals when posts are deflected up to 300 μm. The orientation relative to the earth was found to slightly alter the response of the sensors, which is likely due to a move away from the linear range of the sensors while the earth's magnetic field was oriented against the stray magnetic field of the post. The sensors had a linear voltage-position response in multiple orientations with slightly different sensitivities.

### Frequency and Force Plotting

In testing a representative system, voltage outputs from six sensors simultaneously tracked the active force generation of the posts in the first row of a 24-well plate. Data was recorded using LabView and displayed on screen in real-time during experiments. Post-hoc analysis was performed on all experiments to assess the frequency and magnitude of pulses over time. The data was filtered with a low-pass filter to remove measurement noise using an 8th order Butterworth filter with a cut off frequency of 7 Hz. Peak-to-peak amplitudes were recorded, and the mean was removed from the data. An exponential moving average filter with alpha=0.0001 was used to eliminate the means of the data and account for any low-frequency drift in the system that was not eliminated with the analogue high-pass filters.

After filtering the data, a custom peak finding program found the maximums and minimums of the data with a minimum amplitude of 7 mV, or about 5-10% of the typical baseline motion. Frequency was determined based on the time between maximums. The instantaneous magnitude was determined by subtracting the maximum from the adjacent minimum as long as the maximum and minimum were within two seconds of each other. Data was then grouped together for analysis with 30 seconds grouped at a time. The means of the frequency and magnitudes were averaged over each 30 second period until the end of the experiment at 180 seconds. In order to reduce errors due to slight adjustment of the posts during fluid addition, a four-second window around each fluid addition time point was removed from the averages for both the frequency and magnitude measurements.

### Pharmacological Inhibitors

In embodiments of the present disclosure, tissue specimens affixed to posts and disposed within wells of a multi-well plate may be exposed to therapeutic agents. For example, inhibition experiments may be performed using verapamil hydrochloride (CAS 152-11-4, Tocris Bioscience, Bristol, UK) and isoproterenol hydrochloride (CAS 5984-95-2, Sigma-Aldrich, St. Louis, Mo.). Cardiac tissues may be treated with a mixture of the relevant drug and deionized water. Therapeutic agents may be filtered and portioned into the appropriate dilution based on the final concentration. In some example experiments, some wells may serve as controls having no therapeutic agents.

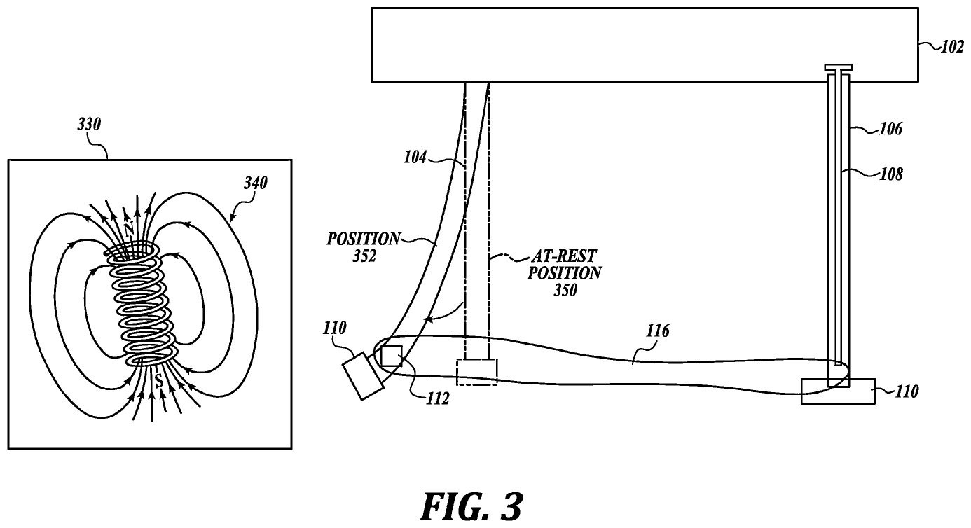

FIG. 3 depicts the example device of FIG. 1 including an external magnet 330 used to simulate preload and afterload in cardiac tissues, in accordance with embodiments of the present disclosure. External magnetic fields may be applied to posts 104 and 106 of device 100 via an external magnet 330. In some embodiments, external magnet 330 may comprise a permanent magnet, while in other embodiments, external magnet 330 may comprise an electromagnetic coil 340 to impart a magnetic force that acts on the magnetic material 112 and pulls against the tissue specimen 116 affixed to post 104 and/or 106. In some embodiments, external magnet 330 may be disposed adjacent to post 104 and/or post 106. External magnet 330 may be effective to attract magnetic material 112 with a first force and thereby displace a distal end of post 104.

When the tissue specimen 116 relaxes during diastole, the restoring force of the post (e.g., a flexible post 104) causes the tissue specimen 116 to stretch like the elastic recoil of the myocardium. However, when a magnetic force is applied, the tissue specimen 116 is stretched further, similar to ventricular expansion during diastolic filling (sometimes referred to as “preload”). Accordingly, the tissue specimen 116 may be strained by a first amount during and/or just after diastole of the cardiac cycle to stress the tissue specimen 116 in order to simulate preload. For example, a flexible post 104 to which tissue specimen 116 is adhered to may stretch from an at-rest position 350 to a position 352 due to the magnetic force from external magnet 330 attracting magnetic material 112.

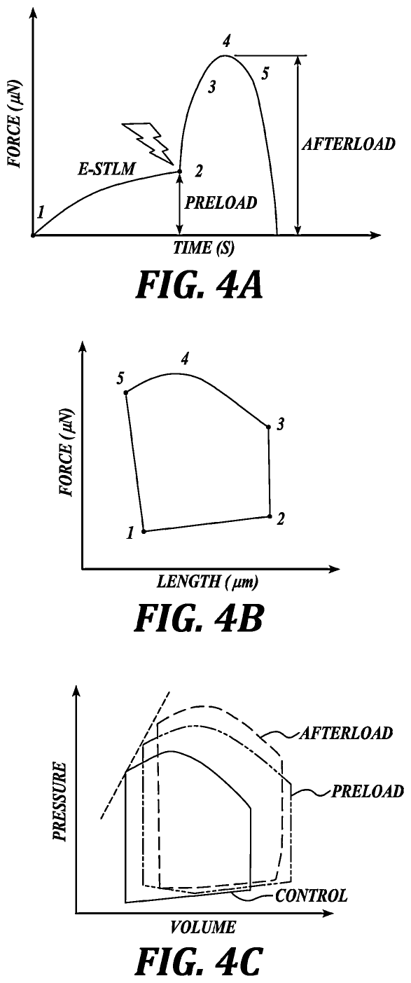

When tissue specimen 116 begins to contract forcibly with electrical stimulation, and at the same time an increase in the magnetic force from external magnet 330 is applied, there is increased resistance to shortening akin to the resistance due to blood pressure during systole (sometimes referred to as “afterload”). Increasing preload and afterload can be done independently and with complex patterns by, for example, controlling the current running through the electromagnetic coil 340 at various points during the cardiac cycle of tissue specimen 116 or by varying the distance between an external magnet 330 and magnetic material 112 (e.g., FIG. 4A).

Using this pattern, the cardiac tissue's length-force (e.g., FIG. 4B) can be compared to cardiac pressure-volume (PV) loops for increasing preload and/or afterload (e.g., FIG. 4C). Thus, preload can be gradually increased to mimic increasing venous return and afterload to mimic increasing blood pressure during development, and change their loading conditions to mimic PV loops of heart failure or hypertension. In various embodiments, the amount of magnetic force applied to the magnetic material 112 may be modulated by changing an amount of current supplied to the electromagnetic coil 340.

### Magnetic Sensors

In various embodiments, high-speed optical microscopy may be used to track post deflections, but such an approach may have low throughput: one sample/well at a time. Additionally, optical techniques may require a large amount of processing resources and data storage as well as expensive optical equipment. Moreover, the image analysis algorithms are cumbersome and require user-input to ensure accuracy of results.

Accordingly, the magnetic approach described herein may be used to record the post deflections using giant magnetoresistive (GMR) sensors and/or other magnetometers (e.g., displacement sensor 120 depicted in FIG. 1). Arrays of GMR sensors and/or other magnetometers may be used, with reference canceling circuits and instrumentation amplifiers for signal processing, to allow for parallel processing of multiple multi-well plates. When tissue specimen 116 pulls on a flexible post (e.g., post 104 from FIG. 1), its movement causes the neodymium magnet or other magnetic material 112 to rotate and translate which, in turn, changes the strength of the magnetic field at the GMR sensor. The change in the field is very small (microteslas) and affects only a local region within its well, and not at the other wells.

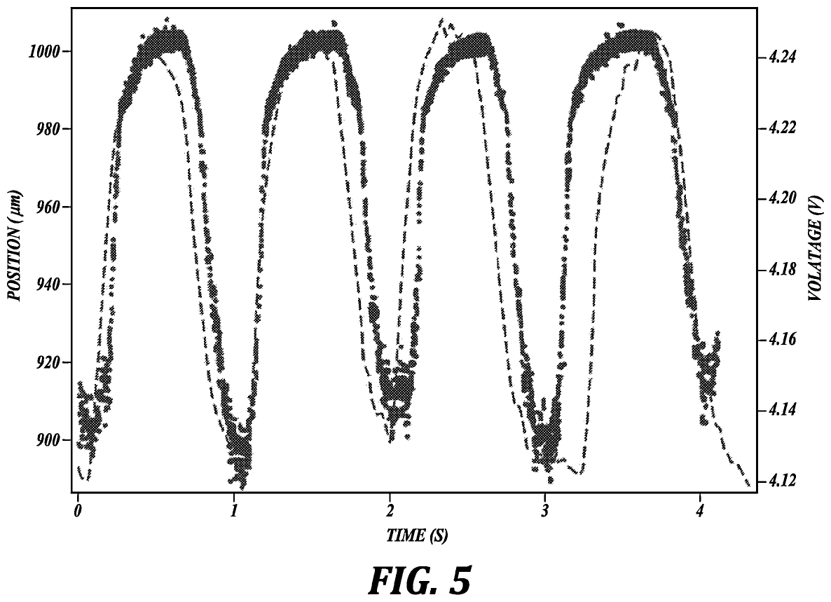

The field change results in a change in voltage output from the sensor, which can be calibrated to correspond to the post deflection detected using optical microscopy, and the corresponding contractile force (see FIG. 5). The voltage signal may be used to determine the force exerted by the tissue specimen 116. Thus, real-time analysis of cardiac tissue contractions is possible without requiring powerful processing and large amounts of data storage. In various embodiments, a PIO controller may be used in conjunction with the electromagnetic coil 340 (FIG. 3) to modulate preload and afterload in response to contractile forces of the tissue specimen 116.

### Preload Effect on Maturation in Tissue Specimen

The following description describes an experiment to simulate preload effect on cultured cardiac tissue in the system for magnetic detection of myocardial forces described herein. The values described below may be altered in different implementations according to the desired strain to be introduced to the cardiac tissue.

Engineered heart tissues may be cultured as previously described, but with minor modifications in order to incorporate the neodymium magnet in the flexible posts. Briefly, 4×105 hiPSC-CMs (cardiomyocytes) may be mixed with 2×10⁵ normal human dermal fibroblasts (a ratio previously optimized) in a fibrin scaffold. Constructs may be allowed to equilibrate for 7 days in order to form a tissue, or until spontaneous beating is observed. From this point on, tissues may be paced at 2 Hz. In this example, the target for preload (circumferential) stretch is based on previous measurements of human LV chamber dimensions during gestation. With electrical stimulation (2 Hz), cardiac tissue may be subjected to continuously increasing magnetic fields of 2% per day over two weeks. This will result in approximately 30% strain, which may be held for an additional 1 week. Strain will be achieved by the applied magnetic field produced by current driven through an electromagnetic coil 340 (FIG. 3), as previously described, and monitored with the GMR sensors, such as displacement sensor 120 depicted in FIG. 1. Using a 24-well format, preload experiments MAY be run in parallel to examine a preload stretch range of 0% to 40% after two weeks.

After conditioning the cardiac tissue with preload, constructs may be fixed and cryo-sectioned for immunohistochemistry to assess their survival and maturation. Constructs may be assessed for proliferation and apoptosis levels, cell size and elongation, myofibril structure (sarcomere spacing and Z-band width by a-actinin), junctional integrity (N-cadherin, connexin-43), T-tubule formation (caveolin-3), β-myosin switching, expression of cTnl and ssTnl (described in Aim 1c.2), electrical maturation (KCNJ2), and ventricular phenotype (MLC2V). Reporter cell lines may be tested for maturation due to preload as they become available from the Allen Institute.

Contractile performance may be assessed biomechanically and conduction velocity assessed via Ca2 imaging (hiPSC-CMs expressing GCaMP6 or Fluo-4). Dynamics of force, velocity, and power may be assessed for constructs as described. Force-length analysis may be conducted in situ by applying magnetically-induced strain on constructs while measuring forces to obtain Frank-Starling curves (end-systolic elastance) and passive stiffness (end-diastolic elastance). Frequency-dependent gain in contractility and kinetics may also be assessed using electrical pacing from 0.5 Hz to 3 Hz (force-Hz response).

### Afterload Effect on Maturation in Tissue Specimen

The following description describes an experiment to simulate afterload effect on cultured cardiac tissue in the system for magnetic detection of myocardial forces described herein. The values described below may be altered in different implementations according to the desired strain to be introduced to the cardiac tissue.

Systolic circumferential tension (afterload) may be estimated on a mid-wall human fetal muscle fiber using published data for LV dimensions and systolic blood pressure during gestation and analysis using Lame's equation. Such an analysis reveals that afterload increases linearly from 2.3 kPa to 8.2 kPa between 10 and 40 weeks. Thus, afterload may be tuned to recapitulate this dynamic range for initial experiments. The P1D controller may be used to ensure that the afterload is not overdriven and to prevent the shortening of cardiac tissue.

Cardiac tissue cultured on the posts without applied preload or afterload can contract with an active force up to 500 μN after 3 weeks. This converts to a longitudinal stress of 2.5 kPa (using an average tissue cross section value of 0.2 mm², which is equivalent in magnitude to the tension due to afterload at 10 weeks. The procedure may begin with zero afterload and may be progressively increased at a rate of 120 μN per day over two weeks. This may result in approximately 1650 μN, which can be held for an additional week. Using a 24-well format, afterload experiments may be run in parallel to examine a range of forces 70% below and 20% above the target of 1650 μN. The survival and maturation of constructs may be assessed as described above. Functional assessment may also be performed for conduction velocities, twitch force, velocity, and power, force-length response, force-frequency response, and tissue elasticity.

### Bioreactor for Combined Preload and Afterload Creep

Since both preload and afterload are continually changing during fetal development, i.e., creep, a combination of preload and afterload may be applied that produces a force-length loop resembling the pressure-volume loops that promote cardiac hypertrophy (FIG. 4C). Using 24-well plates, each cardiac tissue disposed between two microposts may be given a different ratio of preload and afterload. Preload stretch may be increased progressively by 2% and afterload tension increased by 120 μN per day over two weeks. Afterwards, constructs may be assessed for hallmarks of maturation and improved contractile function as described above. Additionally, the combined biomechanical loading may be assessed with a thyroid hormone, triiodothyronine, or the Let-7 transgene on hiPSC-CM maturation and contractile performance

Although in the description above, cardiac tissue is generally described as being cultured between the posts of device 100 (FIG. 1), in various other embodiments, different tissues may be used in accordance with the present disclosure. For example, other tissues that exhibit a rhythmic contraction may be studied using the systems for magnetic detection of forces described herein. Representative tissues include muscle tissue (e.g., engineered heart tissue, smooth muscle tissue, and skeletal muscle tissue) and non-muscle tissue (e.g., ligament tissue and suture tissue). In some embodiments, the systems described in the present disclosure are optimal for detecting and determining forces related to the rhythmic beating of cardiac tissue, as cardiac tissue contracts with a relatively stable frequency.

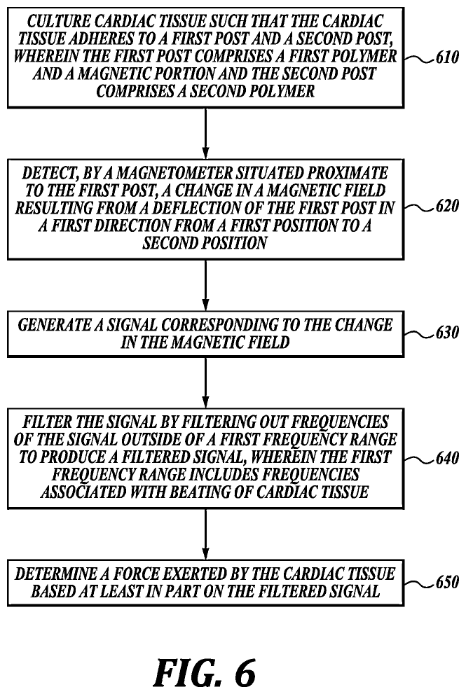

FIG. 6 depicts an example method for magnetically determining force exerted by a tissue specimen, in accordance with various aspects of the present disclosure. The illustrated method is described in the context in the cardiac tissue; however, it shall be appreciated that the following method is applicable to other tissue specimens, including both muscle tissue and non-muscle tissue. Those portions of FIG. 6 that have been described previously with respect to FIGS. 1-5 may not be described again for purposes of clarity and brevity.

The process in FIG. 6 may begin at action 610, “Culture cardiac tissue such that the cardiac tissue adheres to a first post and a second post, wherein the first post comprises a first polymer and a magnetic portion and the second post comprises a second polymer”. At action 610 cardiac tissue may be cultured in such a way that the tissue adheres to a first and a second post. As described above, in some cases the first post may be relatively flexible so as to bend under the force exerted by the cardiac tissue during contraction. Further, in some cases the second post may be relatively rigid so as not to deflect, or so as to minimize deflection during contractions of the cardiac tissue. In some further embodiments, rigidity may be imparted to the second post by inserted a rigid insert within the second post. For example, a silica, glass, plastic, polymeric or other non-magnetically active insert may be placed inside the second post to impart rigidity. In some embodiments, the first post and/or the second post may comprise a magnetic material, such as an earth magnet, embedded within the material comprising the post. For example, a 1 mm3 neodymium magnet may be embedded within the tip of the first post. The posts may comprise a polymer, such as PDMS polymer.

The process in FIG. 6 may continue from action 610 to action 620, “Detect, by a magnetometer situated proximate to the first post, a change in a magnetic field resulting from a deflection of the first post in a first direction from a first position to a second position.” At action 620, a magnetometer (such as displacement sensor 120 depicted in FIG. 1) may be situated proximate to the first, flexible post. The magnetometer may be effective to detect a change in a magnetic field resulting from deflection of the first post as the first post may include a magnetic material, as described herein. In various embodiments, the first post may be flexible and may be deflected by contraction of the cardiac tissue adhered to the first post and disposed between the first post and the second post.

The process in FIG. 6 may continue from action 620 to action 630, “Generate a signal corresponding to the change in the magnetic field.” At action 630, a signal may be generated by the magnetometer in response to the changing magnetic field. For example, in cases where the magnetometer is a GMR sensor, a voltage of the signal generated by the GMR sensor may be modulated by the changes in the magnetic field detected by the GMR sensor. The changes in the magnetic field detected by the GMR sensor may result from movement of the magnet embedded within the flexible first post due to the contraction of the cardiac tissue adhered to the first and second posts.

The process in FIG. 6 may continue from action 630 to action 640, “Filter the signal by filtering out frequencies of the signal outside of a first frequency range to produce a filtered signal, wherein the first frequency range includes frequencies associated with beating of cardiac tissue.” At action 640 the signal generated by the magnetometer (e.g., a GMR sensor or other magnetic sensor) may be filtered by filtering out frequencies of the signal outside of a first frequency range. The first frequency range may be a frequency or a range of frequencies associated with the beating of the cardiac tissue adhered to the first and second post.

Accordingly, magnetic field noise resulting from temperature fluctuation and/or ambient magnetic fields in the local environment may be filtered out and the magnetic field change resulting from the beating of the cardiac tissue may be detected. The cutoff frequencies of the filter used may reflect the expected range of frequencies of the cardiac tissue under observation. Additionally, in some embodiments, the filters may be designed in order to filter out noise resulting from the particular environment. For example, various frequencies of unwanted noise may be produced in the local environment due to machinery and/or other ambient conditions. The particular filters used may be designed to maximize the signal to noise ratio for the particular environment and conditions.

The process in FIG. 6 may continue from action 640 to action 650, “Determine a force exerted by the cardiac tissue based at least in part on the filtered signal.” At action 650, a force exerted by the cardiac tissue may be determined based at least in part on the signal output by the magnetometer and filtered by the electronic frequency filters. Calculations used to determine the force exerted by the cardiac tissue may be performed by a data acquisition device as described above in FIG. 1. Additionally, data generated by the magnetometers and calculated by the data acquisition device may be stored in a memory. Accordingly, the embodiments described herein may allow for real-time and massively parallel monitoring of cardiac tissue with minimal data storage and processing requirements relative to optical techniques for monitoring cardiac tissue.

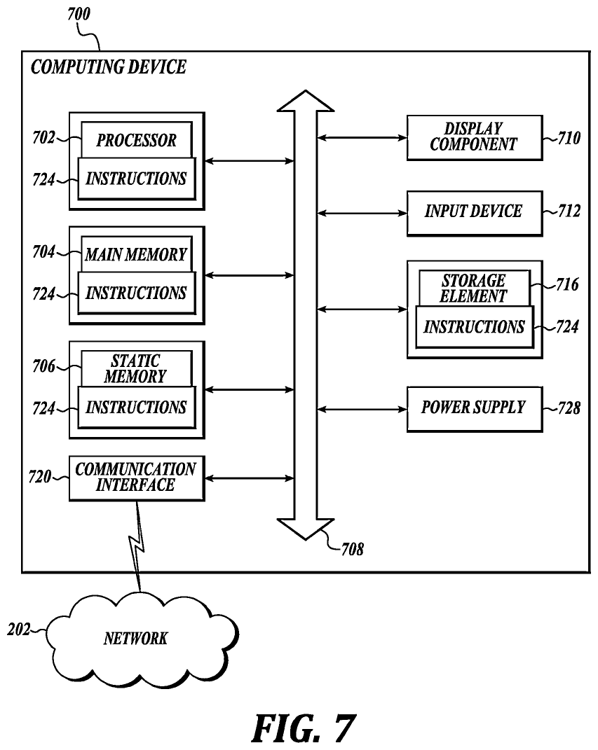

Referring to FIG. 7, the block diagram illustrates components of a computing device 700, according to some embodiments, configured to read instructions 724 from a non-transitory machine-readable storage medium (e.g., a hard drive storage system) and perform any one or more of the methodologies discussed herein, in whole or in part. Specifically, FIG. 7 shows the computing device 700 in the example form of a computer system within which the instructions 724 (e.g., software, a program, an application, an applet, an app, or other executable code) for causing the computing device 700 to perform any one or more of the methodologies discussed herein may be executed, in whole or in part. For example, the computing device 700 may be effective to execute all or a part of the method described above in reference to FIG. 6. Additionally, in some embodiments, the computing device may perform the functions of the data acquisition system described above with respect to FIG. 1.

In some embodiments, the computing device 700 operates as a standalone device or may be connected (e.g., networked) to other computing devices. In a networked deployment, the computing device 700 may operate in the capacity of a server computing device or a client computing device in a server-client network environment, or as a peer computing device in a distributed (e.g., peer-to-peer) network environment. The computing device 700 may include hardware, software, or combinations thereof, and may, as example, be a server computer, a client computer, a personal computer (PC), a tablet computer, a laptop computer, a netbook, a cellular telephone, a smartphone, a set-top box (STB), a personal digital assistant (PDA), a web appliance, a network router, a network switch, a network bridge, or any computing device capable of executing the instructions 724, sequentially or otherwise, that specify actions to be taken by that computing device. Further, while only a single computing device 700 is illustrated, the term “computing device” shall also be taken to include any collection of computing devices that individually or jointly execute the instructions 724 to perform all or part of any one or more of the methodologies discussed herein.

The computing device 700 includes a processor 702 (e.g., a central processing unit (CPU), a graphics processing unit (GPU), a digital signal processor (DSP), an application specific integrated circuit (ASIC), a radio-frequency integrated circuit (RFIC), or any suitable combination thereof), a main memory 704, and a static memory 706, which are configured to communicate with each other via a bus 708. The processor 702 may contain microcircuits that are configurable, temporarily or permanently, by some or all of the instructions 724 such that the processor 702 is configurable to perform any one or more of the methodologies described herein, in whole or in part. For example, a set of one or more microcircuits of the processor 702 may be configurable to execute one or more modules (e.g., software modules) described herein.

The computing device 700 may further include a display component 710, such as one or more devices such light emitting diode (LED) display screens, liquid crystal display (LCD) screens, gas plasma-based flat panel displays, LCD projectors, or other types of display devices.

The computing device 700 may include one or more input devices 712 operable to receive inputs from a user. The input devices 712 can include, for example, a push button, touch pad, touch screen, wheel, joystick, keyboard, mouse, trackball, keypad, accelerometer, light gun, game controller, or any other such device or element whereby a user can provide inputs to the computing device 700. These input devices 712 may be physically incorporated into the computing device 700 or operably coupled to the computing device 700 via wired or wireless interface. For computing devices with touchscreen displays, the input devices 712 can include a touch sensor that operates in conjunction with the display component 710 to permit users to interact with the image displayed by the display component 710 using touch inputs (e.g., with a finger or stylus). In some embodiments, the displacement sensor 120 and/or filter 130 described above with respect to FIG. 1 may be embodiments of input devices 712 operable to provide inputs to computing device 700.

The computing device 700 may also include at least one communication interface 720, comprising one or more wireless components operable to communicate with one or more separate devices within a communication range of the particular wireless protocol. The wireless protocol can be any appropriate protocol used to enable devices to communicate wirelessly, such as Bluetooth, cellular, IEEE 802.11, or infrared communications protocols, such as an IrDA-compliant protocol. It should be understood that the communication interface 720 may also or alternatively comprise one or more wired communications interfaces for coupling and communicating with other devices.

The computing device 700 may also include a power supply 728, such as, for example, a rechargeable battery operable to be recharged through conventional plug-in approaches or through other approaches, such as capacitive charging. Alternatively, the power supply 728 may comprise a power supply unit which converts AC power from the power grid to regulated DC power for the internal components of the device 700.

The computing device 700 may also include a storage element 716. The storage element 716 includes the machine-readable medium on which are stored the instructions 724 (logic) embodying any one or more of the methodologies or functions described herein. The instructions 724 may also reside, completely or at least partially, within the main memory 704, within the processor 702 (e.g., within the processor's cache memory), or both, before or during execution thereof by the computing device 700. The instructions 724 may also reside in the static memory 706.

Accordingly, the main memory 704 and the processor 702 may also be considered machine-readable media (e.g., tangible and non-transitory machine-readable media). The instructions 724 may be transmitted or received over a network 202 via the communication interface 720. For example, the communication interface 720 may communicate the instructions 724 using any one or more transfer protocols (e.g., HTTP).

The computing device 700 may be implemented as any one or more of a number of electronic devices, such as a server, a tablet computing device, a smartphone, a media player, a portable gaming device, a portable digital assistant, a laptop computer, or a desktop computer. In some embodiments, the computing device 700 is a distributed computing device, e.g., a cloud computing device distributed across a plurality of disparate servers. In some example embodiments, the computing device 700 may have one or more additional input components (e.g., sensors or gauges) (not shown). Embodiments of such input components include an image input component (e.g., one or more cameras), an audio input component (e.g., a microphone), a direction input component (e.g., a compass), a location input component (e.g., a GPS receiver), an orientation component (e.g., a gyroscope), a motion detection component (e.g., one or more accelerometers), an altitude detection component (e.g., an altimeter), and a gas detection component (e.g., a gas sensor). Inputs harvested by any one or more of these input components may be accessible and available for use by any of the modules described herein.

As used herein, the term “memory” refers to a non-transitory machine-readable medium capable of storing data temporarily or permanently and may be taken to include, but not be limited to, random-access memory (RAM), read-only memory (ROM), buffer memory, flash memory, and cache memory. The machine-readable medium is non-transitory in that it does not embody a propagating signal. While the machine-readable medium is described in example embodiments as a single medium, the term “machine-readable medium” should be taken to include a single medium or multiple media (e.g., a centralized or distributed database, or associated caches and servers) able to store instructions 724. The term “machine-readable medium” shall also be taken to include any medium, or combination of multiple media, that is capable of storing the instructions 724 for execution by the computing device 700, such that the instructions 724, when executed by one or more processors of the computing device 700 (e.g., processor 702), cause the computing device 700 to perform any one or more of the methodologies described herein, in whole or in part. Accordingly, a “machine-readable medium” refers to a single storage apparatus or device as well as cloud-based storage systems or storage networks that include multiple storage apparatus or devices. The term “machine-readable medium” shall accordingly be taken to include, but not be limited to, one or more tangible (e.g., non-transitory) data repositories in the form of a solid-state memory, an optical medium, a magnetic medium, or any suitable combination thereof.

### Reference Canceling Architecture

Any of the tissue analysis devices and methods described herein may utilize a reference canceling architecture that isolates a first portion of the displacement signal corresponding to the displacement of a post from a second portion of the displacement signal corresponding to an ambient environment in which the device is disposed. Thus, the reference canceling architecture enables the tissue analysis devices of the present disclosure to more accurately determine characteristics of a tissue specimen.

As one example, described below, a tissue analysis device employs a reference canceling architecture instead of a frequency filtering architecture (i.e., instead of a low pass filter, a high pass filter, and/or a bandpass filter), thus preserving an entire frequency range represented in the displacement signal (e.g., a voltage) for at least a portion of the signal processing operations. Thus, frequencies correlative to characteristics of the tissue specimen are preserved even though those frequencies may also reflect noise from the ambient environment. Accordingly, a characteristic determination of the tissue specimen, e.g., a force determination, includes such preserved frequencies. Rather than eliminating ambient noise by filtering out such frequencies, the device cancels a portion of the determined characteristic corresponding to the ambient environment.

Advantageously, the reference canceling architecture described herein enables determination of an absolute force exerted by the tissue specimen. By comparison, tissue analysis devices that utilize a frequency filtering architecture are unable to determine an absolute force exerted by the tissue specimen because such devices filter out “noisy” frequencies, even though such frequencies correlate to force exerted by the tissue specimen.

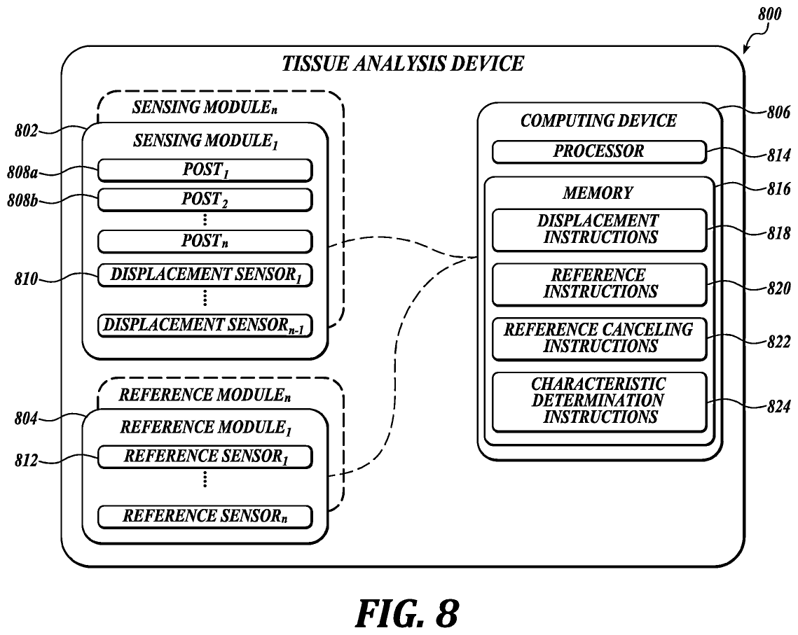

FIG. 8-FIG. 14 show representative tissue analysis devices and methods utilizing reference canceling architectures.

FIG. 8 shows a tissue analysis device 800 in accordance with a representative embodiment of the present disclosure. The tissue analysis device 800 may incorporate any of the features of embodiments described above except where expressly stated herein.

The tissue analysis device 800 includes at least one sensing module 802, at least one reference module 804, and at least one computing device 806. As described below, the sensing module 802 and reference module 804 are communicatively coupled with the computing device 806 such that, when a tissue specimen is affixed to (e.g., adhered to) elements of the sensing module 802 and manipulated (e.g., stimulated by an electrical current), the tissue analysis device 800 determines a characteristic of the tissue specimen that cancels out (i.e., adjusts for) signals caused by the ambient environment. As one representative example, the tissue analysis device 800 determines an absolute force exerted by a tissue specimen in such a manner.

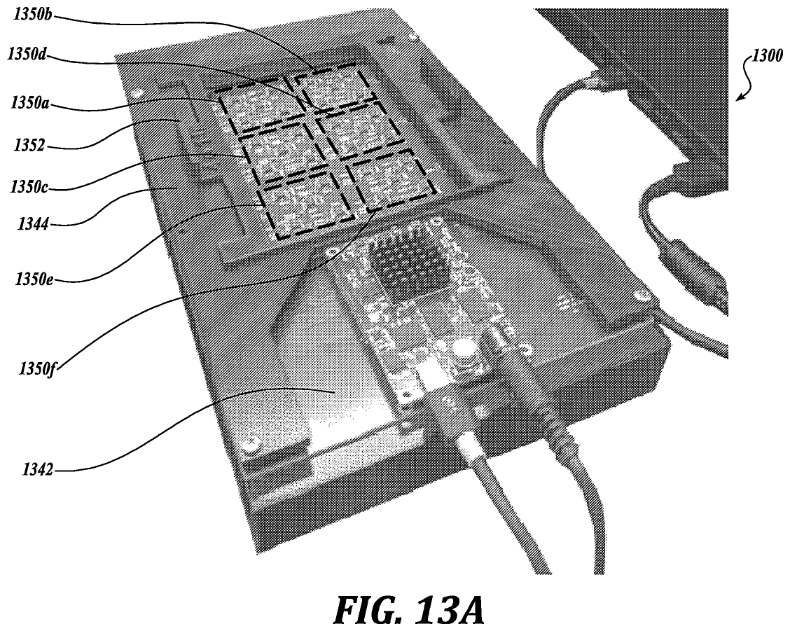

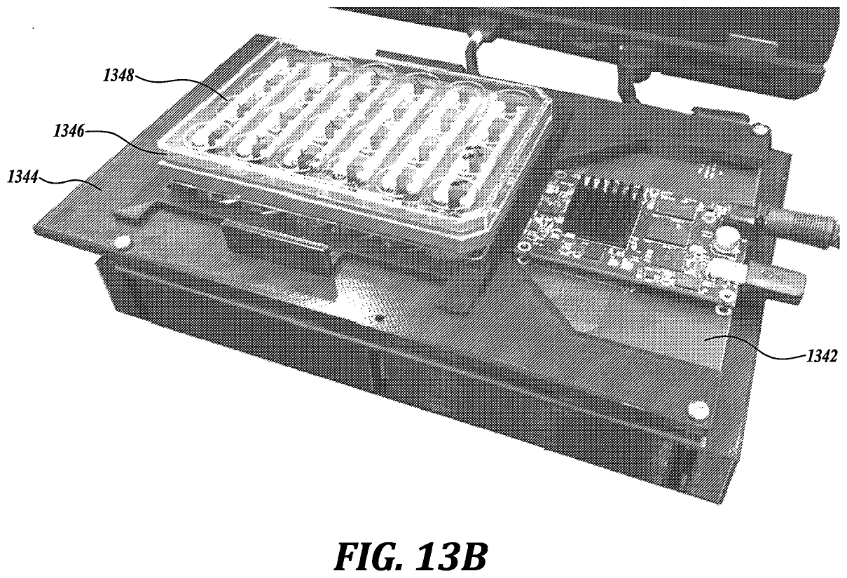

Although shown as distinct modules in FIG. 8 to facilitate understanding, physical elements of the sensing module 802, reference module 804, and computing device 806 may be interspersed among each other. In one embodiment, the tissue analysis device 800 is embodied in a multi-piece assembly comprising a permanent base portion and a disposable culture dish having one or more culture wells. See FIG. 13A-B. In such embodiments, elements of the sensing module 802 are disposed on the culture dish and on a printed circuit board disposed on the base portion, elements of the reference module 804 and computing device 806 are disposed on the printed circuit board, and elements of all three are interspersed among each other on the printed circuit board.

Sensing module 802 includes at least one sensing circuit as described below, and a plurality of posts disposed on a base (such as a base of a well of a culture dish), each post being configured to have a tissue specimen affixed thereto, such as described above with respect to FIG. 1 and FIG. 3. See base 102 and posts 104, 106 of FIG. 1. In this embodiment, sensing module 802 includes two posts 808a-b; thus, sensing module 802 is configured to operate with a single tissue specimen affixed to post 808a and post 808b. However, this number of posts is representative, not limiting. Some embodiments include n posts, i.e., three, four, five, or more posts.

As described above, at least one of the posts is configured to be displaced, such as through bending or deflection in response to a contraction or stretching of a tissue specimen affixed thereto. The terms “displacing post,” “deflecting post,” and “bending post” are used interchangeably herein. In this embodiment, post 808a is configured to be displaced and has magnetic material disposed in its tip, as described above. Likewise, as described above, at least one post may be rigid, i.e., configured such that it is not displaced (i.e., does not bend or deflect) when a tissue specimen affixed thereto contracts or is stretched. In this embodiment, post 808b is rigid.

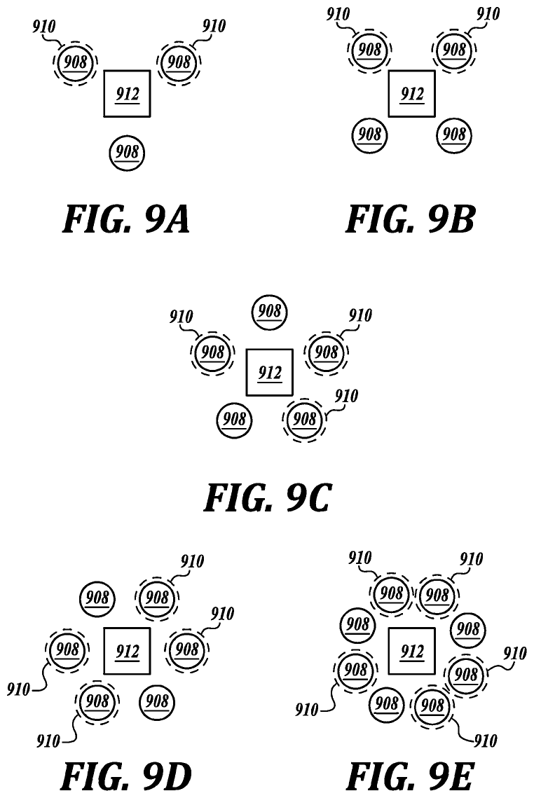

Referring briefly to FIGS. 9A-9E, the posts of the sensing modules described herein may have any number of configurations when disposed on a base, depending on the number of posts provided. For example, an embodiment having at least three posts 908 may have triangular arrangement as shown in FIG. 9A. Likewise, an embodiment having at least four posts 908 may have rectangular arrangement as shown in FIG. 9B. Likewise, an embodiment having at least five posts 908 may have pentagonal arrangement as shown in FIG. 9C. Likewise, an embodiment having at least six posts 908 may have hexagonal arrangement as shown in FIG. 9D. Likewise, an embodiment having at least eight posts 908 may have octagonal arrangement as shown in FIG. 9E. Such post arrangements advantageously enable a plurality of posts to share a common reference sensor (as described below), for example when at least a plurality of posts are positioned equidistant from a centrally-located reference sensor 912. To clarify, the reference sensor 912 need not be equidistant from the posts 908. In any of the foregoing post arrangements, one or more of the posts may be configured to be flexible, and one or more of the posts may be configured to be rigid.

Referring back to FIG. 8, sensing module 802 includes at least one displacement sensor configured to output a displacement signal corresponding to a displacement of one of the posts, such as by sensing a change in a local magnetic field caused by a movement of a magnetic material disposed in a tip of the displaced post. The embodiment of FIG. 8 includes one displacement sensor 810a, which corresponds to post 808a. That is, displacement sensor 810a is configured to output a displacement signal corresponding to a displacement of post 808a.

Positioning of the displacement sensor relative to the posts is discussed below. In some embodiments, the tissue analysis device 800 includes as many displacement sensors as the number of posts configured to be displaced (i.e., bend or deflect). In FIG. 8, because post 808a is configured to bend/deflect but post 808b is rigid, the sensing module 802 includes a single displacement sensor 810a. In some embodiments, the sensing module 802 has n posts and n-1, n/2, and/or n-r displacement sensors (where r corresponds to the number of rigid posts). For example, some embodiments have n-1, n-2, n-3, or n/2 displacement sensors.

Representative displacement sensors include any of those described above, including giant magnetoresistive (GMR) sensors, flux gates, Hall sensors, anisotropic magnetic resistance magnetometers (AMR) sensor, or similar magnetometers. See, e.g., displacement sensor 120 of FIG. 1. As noted above, each displacement sensor is configured to output a displacement signal, which may be an analog or digital signal. The embodiments described herein generally describe a displacement signal as a voltage; however, this is not limiting.

Reference module 804 includes at least one reference circuit, which includes at least one reference sensor 812a configured to output a reference signal corresponding to a reference input, for example an ambient magnetic field. It is expected that the reference input varies due to environmental factors, but not due to the displacement of any of the posts. Accordingly, the reference sensor(s) is positioned far enough away from the posts that the reference input is not disturbed by displacement of any posts. Restated, the reference sensor 812a does not sense any signal amplitude from the displacement of any of the posts 808a-b, due to its placement and sensitivity. Generally, the reference sensor 812a is positioned at least 5-10 mm away from the displacing post 808a and any other displacing posts.

As part of the reference canceling architecture, a reference value based upon the reference input is canceled from a displacement value, which is based upon the displacement of the post(s). Accordingly, the reference sensor is a magnetometer like the displacement sensors. In some embodiments, the reference sensor is a same sensor type as any one or all of the displacement sensors, such as a GMR sensor, flux gate, Hall sensor, or AMR sensor. Although the reference sensor 812a can be a same sensor type as any of the posts 808a-b, it need not have the same sensitivity or other specification.

The reference module 804 of FIG. 8 includes a single reference sensor 812a corresponding to the single deflecting post 808a. However, some embodiments include a plurality of reference sensors, e.g., n reference sensors corresponding to n posts in the sensor module. Further still, some embodiments include as many reference sensors as the number of deflecting posts. In some embodiments, a single reference sensor is a shared reference sensor. In other words, a reference output of a single reference sensor is utilized to determine a reference-canceled characteristic of a plurality of tissue specimens. This creates efficiencies in the construction and operation of the tissue analysis device 800, and contributes to greater accuracy, since the determined tissue characteristics all rely on a common reference signal. Representative placement of the reference sensor 812a is discussed below.

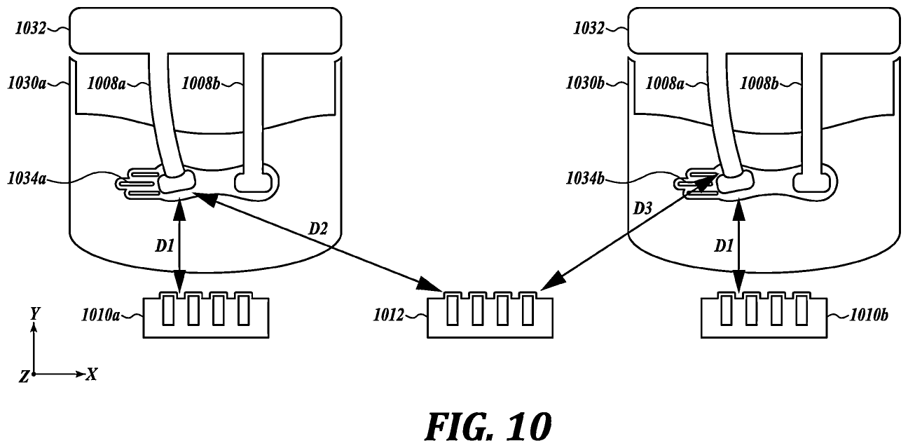

Turning briefly to FIG. 10, one representative arrangement of a tissue analysis device having two sensing modules is illustrated. FIG. 10 shows a cross section elevation view of two wells 1030a, b. Each well includes a base 1032 having two posts 1008a, b disposed thereon. Post 1008a is a deflecting post. Tissue specimens 1034a, b are affixed to the two posts of well 1030a, b, respectively. Displacement sensors 1010a, b are disposed directly beneath the deflecting posts 1008a of wells 1030a, b, at a distance D1 therefrom (e.g., 3 mm-7 mm) that is sufficiently small that displacement sensor 1010a senses a change in local magnetic field due to displacement of post 1008a (of well 1030a), and displacement sensor 1010b senses a change in local magnetic field due to displacement of post 1008a (of well 1030b). D1 corresponds to a length of a vector in three-dimensional space.

As used herein, “directly beneath” means, in the context of a three-dimensional Euclidean space represented by orthogonal x-, y-, and z-axes as shown, that both the displacement sensor 1010a and deflecting post 1008a (of well 1030a) have common x-and z-coordinates (within 1-2 mm), but the displacement sensor 1010a has a lesser y-coordinate than the post 1008a. Likewise, the displacement sensor 1010b and post 1008a (of well 1030b) have common x- and z-coordinates (within 1-2 mm), but the displacement sensor 1010b has a lesser y-coordinate than the post 1008a (e.g., 5-10 mm less). This arrangement enables displacement sensor 1010a to sense a displacement of post 1008a (of well 1030a) due to the contraction or stretching of tissue specimen 1034a, but not tissue specimen 1034b. Likewise, this arrangement enables displacement sensor 1010b to sense a displacement of post 1008a (of well 1030b) due to the contraction or stretching of tissue specimen 1034b, but not tissue specimen 1034a. In some embodiments, rather than being disposed directly beneath the posts, the displacement sensors are disposed directly above or directly to the side of the posts.

Reference sensor 1012 is disposed between well 1030a, b and configured to sense a reference input—in this embodiment, an ambient magnetic field. Reference sensor 1012 is disposed a second distance D2 away from post 1008a (in particular, the magnetic material disposed in a tip thereof), and a third distance D3 away from well 1030b (in particular, the magnetic material disposed in a tip thereof). D2 and D3 both refer to a length of a three-dimensional vector, as with D1. Given a particular sensitivity for reference sensor 1012, both D2 and D3 are sufficiently large that reference sensor 1012 does not sense any signal amplitude due to the displacement of posts 1008a, e.g., 10 mm-20 mm. Accordingly, D2 and D3 are both greater than D1.

In FIG. 10, D2 and D3 are equal, i.e., reference sensor 1012 is disposed equidistant from both displacement sensors 1010a, b and both tissue specimens 1034a, b. However, in some embodiments, D2 and D3 are not equal. For example, in some embodiments, D2 and D3 are not equal, but both D2 and D3 are sufficiently large that reference sensor 1012 does not sense any signal amplitude when either of posts 1008a deflect. For example, in an embodiment, D2=17 mm and D3=11 mm. In this disclosure, “equidistant” means “equidistant in three-dimensional space” (i.e., a first three dimensional vector from reference sensor 1012 to post 1008a has a length D2, which is the same as a length D3 of a second three dimensional vector from reference sensor 1012 to post 1008b).

Referring again briefly to FIGS. 9A-9E, the sensing module of FIG. 9A includes three posts 908, the top two of which are configured to be flexible, and the bottom of which is configured to be rigid. Accordingly, the top two posts 908 each have a corresponding displacement sensor 910 disposed directly beneath. This contemplates that two tissue specimens may be affixed to the rigid bottom post in a “V” configuration. In any of the embodiments described herein, a rigid post may be configured to have more than one tissue specimen affixed thereto. In FIG. 9B, the top two posts 908 are configured to be flexible, and accordingly each has a displacement sensor 910 disposed directly beneath. The bottom two posts are rigid, and therefore the tissue analysis device is configured to have two parallel tissue specimens affixed thereto—one tissue specimen between each flexible post 908 and each rigid post 908. Similarly, the tissue analysis device of FIG. 9C includes three displacement sensors 910, each disposed directly beneath one post 908 which is configured to be flexible; the two posts 908 without displacement sensors 910 are configured to be rigid. Similarly, the tissue analysis device of FIG. 9D includes four displacement sensors 910, each disposed directly beneath one post 908 which is configured to be flexible; the two posts 908 without displacement sensors 910 are configured to be rigid. Similarly, the tissue analysis device of FIG. 9E includes five displacement sensors 910, each disposed directly beneath one post 908 which is configured to be flexible; the three posts 908 without displacement sensors 910 are configured to be rigid. The foregoing embodiments are representative, not limiting. For example, the four-post embodiment of FIG. 9B may have two flexible posts 908 disposed on diagonally opposite sides of the reference sensor 912. As another example, the six-post embodiment of FIG. 9D may have three flexible posts 908 and three displacement sensors 910, instead of four each.

Returning to FIG. 8, computing device 806 can have any of the features of the computing device 700 of FIG. 7. To facilitate understanding, in this embodiment, computing device 806 includes a processor 814 (e.g., a general processing unit, graphical processing unit, and/or application specific integrated circuit); a memory 816 (a tangible machine-readable storage medium); and a plurality of instruction modules that may be implemented as software logic (e.g., executable software code), firmware logic, hardware logic, or various combinations thereof.

Some embodiments of the tissue analysis device 800 exclude certain elements of the computing device 806 shown in FIG. 8. For example, some embodiments of the tissue analysis device 800 include instruction modules as described below, which are configured to operate on an existing computing device; however, the tissue analysis device 800 may not include a computing device per se, i.e., the tissue analysis device 800 does not include the processor 814 and/or memory 816. Thus, some embodiments of the tissue analysis device 800 include at least one sensing module 802, and least one reference module 804, and one or more instruction modules as described below, which may be embodied as software or hardware (e.g., on a printed circuit board or ASIC).

The computing device 806 includes a communications interface having circuits configured to enable communication with the sensing module 802 (in particular, the displacement sensor 810a) and reference module 804 (in particular, the reference sensor 812a), a remote server, a base station, or other network element via the internet, cellular network, RF network, Personal Area Network (PAN), Local Area Network, Wide Area Network, or other network. Accordingly, the communications interface may be configured to communicate using wireless protocols (e.g., WIFI®, WIMAX®, BLUETOOTH®, ZIGBEE®, Cellular, Infrared, Nearfield, etc.) and/or wired protocols (Universal Serial Bus or other serial communications such as RS-234, RJ-45, etc., parallel communications bus, etc.). In some embodiments, the communications interface includes circuitry configured to initiate a discovery protocol that allows the computing device 806 and other network element (e.g., any of the displacement sensors and reference sensors) to identify each other and exchange control information. In an embodiment, the communications interface has circuitry configured to a discovery protocol and to negotiate one or more pre-shared keys.

As used herein, the memory 816 is a tangible machine-readable storage medium that includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.). In some embodiments, the memory 816 is distributed across a plurality of network elements (e.g., remote servers, local computing devices, etc.).

As stated above, the computing device 806 includes a plurality of instruction modules. Each module includes logic (instructions) that, when executed by the processor 814, causes the tissue analysis device 800 to perform one or more operations related to the sensing, detection, measurement, and/or determination of one or more characteristics of at least one tissue specimen affixed to the posts. Although described as discrete instruction modules herein to facilitate understanding, in some embodiments, the logic is embodied in a single module or a different number of modules than shown. Moreover, logic described below with respect to a particular module may, in other embodiments, exist in different modules than described here.

Further still, the instruction modules need not be embodied in a single network element; in some embodiments, the instruction modules are stored and/or executed across a plurality of network elements (e.g., remote servers, local computing devices, etc.). Further still, although the logic is described herein in the context of software, any of the instruction modules described herein can be implemented as firmware, e.g., as circuitry on a printed circuit board. Further still, embodiments of any of the instruction modules described herein include instructions to execute some or all of the instructions continuously, periodically (e.g., every half second, second, minute, hour, or other period), and/or on demand.

To facilitate understanding, embodiments provided below describe instructions in the context of a single tissue specimen corresponding to a single deflecting post 808a and a rigid post 808b. However, this is not limiting. Embodiments of tissue analysis devices described herein have a plurality of sensing modules, and/or a sensing module having a plurality of posts, and thus are configured to determine characteristics of a plurality of tissue specimens. Accordingly, any of the instructions below may be executed in connection with a plurality of tissue specimens, for example in parallel or in series.

Displacement instructions 818 determine one or more displacement values based upon the displacement signal output by the displacement sensor 810a. For a tissue analysis device having n displacement sensors, the displacement instructions 818 determines n displacement values. Exemplary instructions configured to determine a displacement value for a single displacement sensor will now be described; however, it shall be appreciated that such instructions may be executed for all displacement sensors of the tissue analysis device, e.g., in parallel. In some embodiments, the displacement value is denominated in in units of physical translation (e.g., mm); however, in some embodiments, the displacement value is denominated in other units (e.g., microteslas). Displacement instructions 818 determine displacement values continuously, periodically (e.g., every half second, second, minute, hour, or other period), or on demand

In some embodiments, displacement instructions 818 determine a displacement value (e.g., in microteslas) corresponding to a change in local magnetic field caused by the displacement of post 808a, such as caused by the contraction of a tissue specimen affixed between posts 808a, b. In such embodiments, the displacement value is determined by multiplying the displacement signal of the displacement sensor 810a by a first linear factor α₁, which is based upon known relationship between the output of the displacement sensor 810a (e.g., voltage) and the corresponding change in the sensed magnetic field (e.g., in microteslas). The first linear factor α₁ may be valid within a limited range of linear behavior for the particular displacement sensor 810a.

In some embodiments, displacement instructions 818 determine a displacement value (e.g., in mm) corresponding to physical displacement of post 808a (again, such as caused by the contraction of a tissue specimen affixed between posts 808a, b) by multiplying the displacement signal output by displacement sensor 810a by the first linear factor α₁ and by a first non-linear factor β₁. In some embodiments, the first non-linear factor β₁ is based upon an empirically-validated or mathematically-modeled correlation factor between the change in magnetic field and displacement of the post (e.g., in mm). In some embodiments, the first non-linear factor β₁ is a function of the field strength of the magnetic material, a position or orientation of a magnetic material in the post 808a, and/or a position of the post 808a relative to the displacement sensor 810a. It shall be appreciated that in embodiments having more than one displacement sensor, the first linear factor α₁ and first non-linear factor β₁ may differ between displacement sensors. Thus, a tissue analysis device having n displacement sensors can have n first linear factors (e.g., α_{1,1} . . . α_{1,n}) and n first non-linear factors (β_{1,1} . . . β_{1, n}). Of course, in some embodiments, the first linear factor and first non-linear factor are the same for all displacement sensors.

In some embodiments, determining the displacement value does not include frequency filtering or similarly processing the displacement signal(s) prior to multiplying the displacement signal by the first linear factor α1 and/or by the first non-linear factor β₁. For example, in some embodiments, the displacement value is determined as described above—without processing the displacement signal through any high pass, low pass, or band pass filters, or through any similar signal processing techniques, prior to multiplying the displacement signal by the first linear factor α₁ and/or by the first non-linear factor β₁. While frequency filtering the displacement signal can reduce signal noise, it has the consequence of filtering frequencies correlative to characteristics of the tissue specimen, thus preventing the determination of certain tissue characteristics, e.g., absolute force. Accordingly, by determining the displacement value without frequency filtering the displacement signal before multiplying the displacement signal by the first linear factor α₁ and/or the first non-linear factor β₁, the entire displacement signal is preserved. Advantageously, this preserves the ability to determine certain absolute characteristics of the tissue specimen (e.g., absolute force), as described herein. In some embodiments, determining the displacement value includes determining a plurality of displacement value components, each component corresponding to sensed magnetic fields or physical displacement in a plurality of axes. In such embodiments, one or more displacement sensors has a common orientation with one or more reference sensors.

Reference instructions 820 determine a reference value based upon the reference signal output by the reference sensor 812a, which senses the ambient magnetic field without any signal amplitude from the displacement of post 808a. Representative instructions to determine a reference value for a single displacement sensor will now be described; however, it shall be appreciated that such instructions may be executed for n reference sensors of the tissue analysis device, e.g., in parallel.

As part of the reference canceling architecture, the reference value is canceled from the displacement value(s), or from an intermediate value thereof. Accordingly, in any of the embodiments, the displacement value(s) are time-indexed to the reference value(s). In some embodiments, the reference value has the same units as the displacement value(s). For example, in some embodiments, the reference value has units corresponding to a change in ambient magnetic field (e.g., microteslas). In one such embodiment, determining the reference value includes multiplying the reference signal by a second linear factor α₂, which may be the same as or different from the first linear factor α₁ utilized by the displacement instructions 818. For example, if the reference sensor 812a is a different sensor type and/or has different specifications than the displacement sensor 810a, then the first linear factor α₁ and second linear factor α₂ may be different. For example, in some embodiments, the second linear factor α₂ is based upon a known relationship between a voltage output by the reference sensor 812a and the corresponding change in the sensed ambient magnetic field (e.g., in microteslas). The second linear factor α₂ may be valid within a limited range of linear behavior for the particular reference sensor 812a.

In some embodiments, the reference value has units corresponding to the physical displacement of displacement sensor 810a, e.g., mm. In such embodiments, determining the reference value includes multiplying the reference signal by the second linear factor α₂ and by a second non-linear factor β₂, which is a function of the ambient magnetic field strength, and/or a position of the reference sensor 812a. In some embodiments, determining the reference value includes determining a plurality of reference value components, each component corresponding to ambient magnetic fields in a plurality of axes. In such embodiments, one or more reference sensors have a common orientation with one or more displacement sensors.

Reference canceling instructions 822 determine one or more reference-canceled displacement value(s) based upon the displacement value(s) determined by the displacement instructions 818, and based upon the reference value(s) determined by the reference instructions 820. In some embodiments, the reference-canceled displacement value corresponding to the displacement of post 808a is based upon subtracting the reference value from the displacement value. As one example:

Δ_{RC}=Δ_{SM}−Δ_{RM}

where: Δ_{SM}=α₁β₁γ and γ=the displacement signal of displacement sensor 810a; and Δ_{RM}=α₂β₂ε and ε=the reference signal of reference sensor 812a.

In some embodiments, determining the reference-canceled displacement value is based upon subtracting at least one of the plurality of displacement value components from a corresponding one of the plurality of reference value components.

In some embodiments, the reference value(s) determined by the reference instructions 820 are shared, i.e., utilized by the reference canceling instructions 822 to determine reference-canceled displacement values corresponding to a plurality of posts.

Characteristic determination instructions 824 determine one or more characteristics of one or more tissue specimens based upon the reference-canceled displacement value(s) determined by the reference canceling instructions 822 and corresponding to that tissue specimen. As one example, the characteristic determination instructions 824 determine a force exerted by the tissue specimen affixed to posts 808a, b when it is stimulated by an electrical current. The characteristic determination instructions 824 determine the force by multiplying the reference-canceled displacement value by a third linear factor α₃, which may be an empirically-validated or mathematically-modeled correlation factor between post displacement and tissue force (e.g., force exerted by the tissue specimen). For cardiac tissue, such force determinations may correspond to systole or diastole. In some embodiments where the displacement signal is not frequency filtered (e.g., prior to the multiplication by α1 and β1) the determined force is an absolute force (e.g., an absolute myocardial force). Advantageously, the ability to determine an absolute force exerted by a tissue specimen enables doctors and researchers to better assess the maturity and viability of such tissue specimen.

Force is one modality; however, in some embodiments, the characteristic determination instructions 824 determine strain, stress, and/or other characteristic of the tissue specimen. For example, in some embodiments, the characteristic determination instructions 824 determine a strain of the tissue specimen by dividing the reference-canceled displacement value (e.g., in mm) by a reduction in the cross-sectional dimension of the tissue specimen (which may be optically determined or determined through other measurement means). As another example, in some embodiments, the characteristic determination instructions 824 determine a stress of the tissue specimen by first determining the absolute force exerted by or on the tissue specimen, and then dividing the absolute force by a cross-sectional area of the tissue specimen (which may be optically determined or determined through other measurement means). These modalities are representative, not limiting.

The foregoing instruction modules are representative, not limiting. In some embodiments, the computing device 806 includes additional instruction modules, for example a communication module having instructions that, when executed, cause the computing device 806 to transmit the determined tissue characteristics to a remote network element.

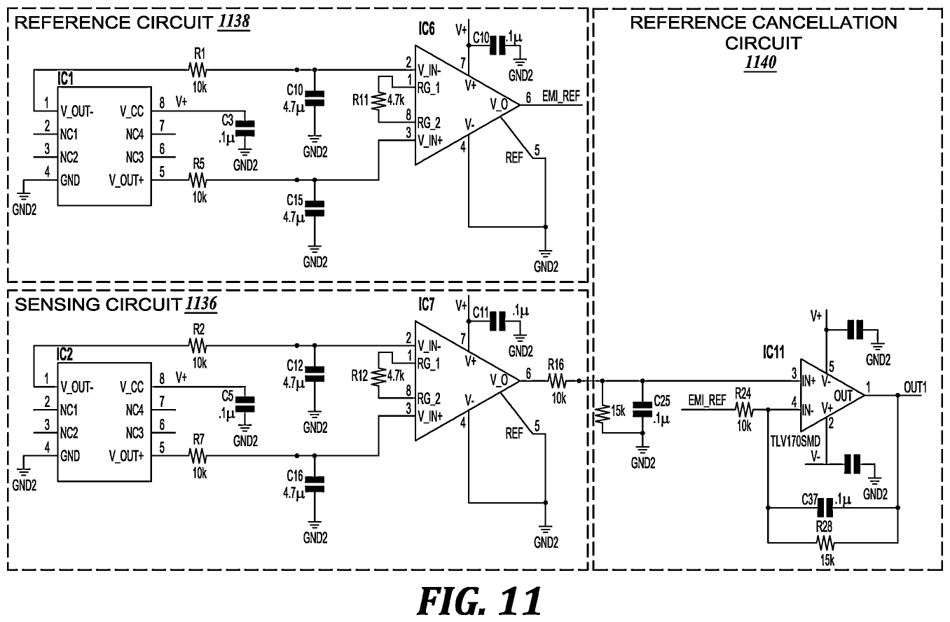

FIG. 11 shows representative circuitry for an embodiment of the tissue analysis device of the present disclosure, which is at least partially embodied on a printed circuit board, and ASIC, or similar device. A sensing circuit 1136 includes at least one displacement sensor (such as displacement sensor 810a of FIG. 8), which is operatively connected to one or more filters and amplifiers. Although the sensing circuit 1136 includes a single displacement sensor in FIG. 11, it shall be understood that some embodiments include a plurality of displacement sensors, as described above. Some embodiments do not include frequency filtering circuitry, as described above. The sensing circuit 1136 forms part of a sensing module as described above (e.g., sensing module 802 of FIG. 8).

A reference circuit 1138 includes at least one reference sensor (such as reference sensor 812a of FIG. 8), which is operatively connected to one or more filters and amplifiers. Although the reference circuit 1138 includes a single reference sensor in FIG. 11, it shall be understood that some embodiments include a plurality of reference sensors, as described above. Some embodiments do not include frequency filtering circuitry, as described above. The reference circuit 1138 forms part of a reference module as described above.

The sensing circuit 1136 and reference circuit 1138 each provide their own displacement value and reference value, respectively. Reference cancelation circuit 1140, in addition or alternatively to the reference canceling instructions described above, cancels the reference value from the displacement value, thereby obtaining a reference-canceled value, e.g., a reference-canceled displacement value. In some embodiments, the reference cancelation circuit 1140 is part of the sensing module, reference module, both, or a separate module.

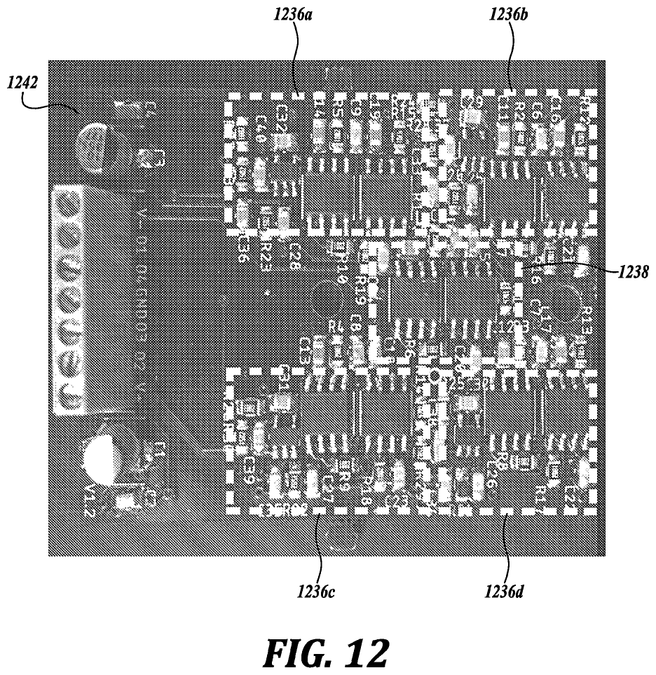

FIG. 12 shows one representative printed circuit board 1242 having circuitry of a tissue analysis device as described herein. In particular, the printed circuit board 1242 includes a sensor array having four sensing circuits 1236a-d and one reference circuit 1238. Each sensing circuit 1236a-d is similar to sensing circuit 1136 of FIG. 11. In this embodiment, the sensing circuits 1236a-d are arranged in a rectangular array. Each sensing circuit 1236a-d is configured to be positioned directly beneath of plurality of posts of a sensing module as described above, such that each sensing circuit detects a change in a local magnetic field caused by a displacement of a post disposed directly above it (e.g., post 808a). Some embodiments have greater or fewer sensing circuits, e.g., one, two, three, five, six, etc. In such embodiments, the sensing circuits may be arranged in any one or more of the configurations described herein, including a triangular, pentagonal, hexagonal, octagonal, or other arrangement.

Printed circuit board 1242 also includes a single reference circuit 1238, similar to that described above with respect to FIG. 11. The reference circuit 1238 is centrally located between, and equidistant from, the sensing circuits 1236a-d. In particular, reference circuit 1238 is equidistant from sensing circuits 1236a-d in three dimensions. That is, the sensing circuits 1236a-d and reference circuit 1238 are disposed on a common plane of the printed circuit board 1242, and reference circuit 1238 is equidistant from each sensing circuit 1236a-d in the plan view of FIG. 12. Such equidistant placement can be utilized in any of the embodiments described herein.

Advantageously, equidistant placement of the reference circuit 1238 enables it to be shared between the sensing circuits 1236a-d, i.e., the reference value determined by the reference circuit 1238 is utilized to determine a reference-canceled displacement value for each of the sensing circuits 1236a-d. Thus, the illustrated embodiment has a 4:1 ratio of sensing circuits to reference circuits. The illustrated embodiment also has a 4:1 ratio of displacement sensors to reference sensors. This ratio is representative, and some embodiments have a lesser or greater ratio, e.g., 2:1, 3:1, 5:1, 6:1, 7:1, or 8:1. Importantly, the reference circuit 1238 (in particular the reference sensor) is positioned far enough away from each sensing circuit 1236a-d such that its reference signal is not disturbed by the displacement of any of the posts disposed over each sensing circuit 1236a-d. In one embodiment, the reference sensor is disposed at least 5 mm away from each displacement sensor (e.g., 5 mm-20 mm). In use, reference circuit 1238 is not disposed directly beneath any posts.

The printed circuit board 1242 is configured for use with a culture dish having a plurality of wells, each well having a plurality of posts as described above. For example, each sensing circuit 1236a-d is configured to be disposed directly beneath one well of the culture dish. Advantageously, printed circuit board 1242 is modular. For example, six printed circuit boards 1242 can be operatively connected in parallel to a computing device (as described above) and simultaneously positioned beneath a 24-well culture dish in order to enable high throughput analysis of tissue specimens. Likewise, a single printed circuit board can be formed with a plurality of the 4:1 arrays shown in FIG. 12, e.g., six such arrays, providing 24 sensing circuits. Of course, this example is representative, not limiting; other embodiments have a different number of sensing circuits and/or reference circuits, and/or a different ratio of sensing circuits to reference circuits.

FIGS. 13A-B show aspects of a representative tissue analysis device 1300 of the present disclosure. The tissue analysis device 1300 includes a permanent base portion 1344 which is configured to be utilized repeatedly, i.e., it is not a consumable assembly. The tissue analysis device 1300 is configured to be utilized in connection with one or more consumable culture dish 1346, each having a plurality of wells 1348, and each well 1348 having a plurality of posts disposed therein with a tissue specimen affixed thereto, as discussed above. See FIG. 13B. In some embodiments, the tissue analysis device 1300 includes the culture dish 1354a.

The base portion 1344 may be constructed from polymer, metals (e.g., one or more ferromagnetic metals), and/or a combination thereof. The base portion 1344 provides a stable foundation for a printed circuit board 1342 having circuitry as described above. Referring to FIG. 13A, the printed circuit board 1342 of the illustrated embodiment has six 4:1 sensor arrays 1350a-f, each being as described above with respect to FIG. 12. That is, each sensor array 1350a-f includes four displacement sensors and a single, centrally-located shared reference sensor disposed equidistant from each displacement sensor of the array (i.e., equidistant in three dimensions as described above). The sensor array configuration shown is representative, not limiting. The printed circuit board 1342 of the illustrated embodiment is configured to communicate with a controller (e.g., a remote computing device) having a processor and memory, which may form part of the tissue analysis device 1300.

An opening in the base portion 1344 formed over the sensor arrays 1350a-f is aligned with a cradle 1352 configured to reversibly hold the culture dish 1346, such that each well is disposed directly over one displacement sensor, but not over any reference sensors. Accordingly, when the culture dish 1346 is disposed in the cradle 1352, the displacement sensors are configured to sense a displacement of a post in each well (e.g., due to a contraction of the corresponding tissue specimen), and the reference sensors are configured to sense an ambient magnetic field.

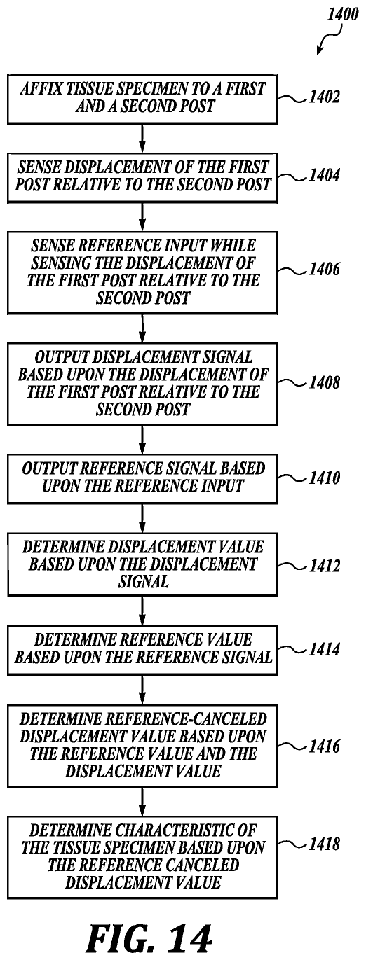

FIG. 14 provides a representative method 1400 for determining a characteristic of a tissue specimen. This method may be practiced by or in connection with the tissue analysis devices of the present disclosure. Accordingly, terms used below with respect to the methods have alike meanings as alike terms used above in connection with the tissue analysis devices.

In block 1402, a tissue specimen is affixed to a first post and a second post, which may be disposed on a base in a well of a culture dish.

In block 1404, a displacement of the first post relative to the second post is sensed, e.g., in response to a stimulation of the tissue specimen with an electrical current. The displacement may be sensed as described above with respect to the tissue analysis device 800 of FIG. 8.

In block 1406, a reference input is sensed while sensing the displacement of the first post relative to the second post. The reference input may be sensed as described above with respect to the tissue analysis device 800 of FIG. 8.

In block 1408, a displacement signal based upon the displacement of the first post relative to the second post is output. The displacement signal may be output as described above with respect to the tissue analysis device 800 of FIG. 8.

In block 1410, a reference signal based upon the reference input is output. The reference signal may be output as described above with respect to the tissue analysis device 800 of FIG. 8.

In block 1412, a displacement value is determined based upon the displacement signal. The displacement value may be determined as described above with respect to the tissue analysis device 800 of FIG. 8.

In block 1414, reference value is determined based upon the reference signal. The reference value may be determined as described above with respect to the tissue analysis device 800 of FIG. 8.

In block 1416, a reference-canceled displacement value based upon the reference value and the displacement value is determined. The reference-canceled displacement value may be determined as described above with respect to the tissue analysis device 800 of FIG. 8.

In block 1418, a characteristic of the tissue specimen based upon the reference-canceled displacement value is determined. In one embodiment, the characteristic is a force (e.g., an absolute force), a strain, or a stress. The characteristic may be determined as described above with respect to the tissue analysis device 800 of FIG. 8.

Thus, the present disclosure provides high throughput tissue analysis devices configured to determine characteristics of tissue specimens. In particular, the tissue analysis devices and methods of the present disclosure enable determination of an absolute force exerted by a tissue specimen, along with other tissue characteristics.

While the invention has been described in terms of particular embodiments and illustrative figures, those of ordinary skill in the art will recognize that the invention is not limited to the embodiments or figures described. For example, in various embodiments described above, a single pair of polymeric posts are described between which a tissue specimen is cultured. However, in other embodiments, an array of post-pairs may be arranged on one or more common bases, with each post-pair having a tissue specimen cultured between, and affixed to, the post-pairs.

The detailed description set forth above in connection with the appended drawings, where like numerals reference like elements, are intended as a description of representative embodiments of the present disclosure and are not intended to represent the only embodiments. Each embodiment described in this disclosure is provided as an example or illustration and should not be construed as preferred or advantageous over other embodiments. The illustrative embodiments provided herein are not intended to be exhaustive or to limit the disclosure to the precise forms disclosed. Similarly, any steps described herein may be interchangeable with other steps, or combinations of steps, in order to achieve the same or substantially similar result. Further still, one or more features of any embodiment may be combined with one or more features of one or more embodiments to form additional embodiments, which are within the scope of the present disclosure.

Generally, the embodiments disclosed herein are non-limiting, and the inventors contemplate that other embodiments within the scope of this disclosure may include structures and functionalities from more than one specific embodiment shown in the FIGURES and described in the specification. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed. For example, the present disclosure includes additional embodiments having combinations of any one or more features described above with respect to the representative embodiments.

In the foregoing description, specific details are set forth to provide a thorough understanding of representative embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure.

The present application may include references to directions, such as “first,” “second,” “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.” The term “between” includes the values recited in connection therewith.

1. A tissue analysis device for determining a characteristic of a tissue specimen, comprising:
a sensing module, comprising:
a first post disposed on a base, having a magnetic material disposed therein, and configured to have the tissue specimen attached thereto;
a second post disposed on the base and configured to have the tissue specimen attached thereto; and
a displacement sensor configured to output a displacement signal corresponding to a displacement of the first post;
a reference module comprising a reference sensor configured to output a reference signal corresponding to a reference input; and
a non-transitory machine readable storage medium storing logic, which when executed by a processor, causes the processor to perform operations, including:
determining a displacement value based upon the displacement signal;
determining a reference value based upon the reference signal;
determining a reference-canceled displacement value based upon the displacement value and the reference value; and
determining the characteristic based upon the reference-canceled displacement value.

2-4. (canceled)

5. The tissue analysis device of claim 1, wherein determining the reference-canceled displacement value is based upon subtracting the reference value from the displacement value.

6. The tissue analysis device of claim 1, wherein determining the displacement value comprises multiplying the displacement signal by a linear factor and by a non-linear factor.

7. The tissue analysis device of claim 6, wherein determining the displacement value does not comprise frequency filtering the displacement signal before multiplying the displacement signal by the linear factor.

8. The tissue analysis device of claim 1, wherein determining the reference value comprises multiplying the reference signal by a linear factor.

9. The tissue analysis device of claim 1, wherein determining the characteristic comprises multiplying the reference-canceled displacement value by a linear factor.

10. The tissue analysis device of claim 9, wherein the linear factor is a correlation factor between the displacement of the first post and a force exerted by the tissue specimen.

11. (canceled)

12. The tissue analysis device of claim 1, wherein the displacement sensor is disposed a first distance away from the first post and the reference sensor is disposed a greater second distance away from the first post.

13. The tissue analysis device of claim 12, wherein the second distance is sufficiently large that the reference sensor does not sense any signal amplitude from the tissue specimen.

14-15. (Canceled)

16. The tissue analysis device of claim 1, wherein the first post and the second post are disposed in a well of a culture dish, and the displacement sensor is disposed directly beneath the first post on a printed circuit board.

17. The tissue analysis device of claim 1, wherein the displacement signal corresponds to a change in a local magnetic field caused by the displacement of the first post, and the reference signal corresponds to an ambient magnetic field.

18-19. (canceled)

20. The tissue analysis device of claim 1, wherein the characteristic is an absolute force.

21. (canceled)

22. A method, comprising:
affixing a tissue specimen to a first post and a second post;
sensing a displacement of the first post relative to the second post;
sensing a reference input while sensing the displacement of the first post relative to the second post;
outputting a displacement signal based upon the displacement of the first post relative to the second post;
outputting a reference signal based upon the reference input;
determining a displacement value based upon the displacement signal;
determining a reference value based upon the reference signal;
determining a reference-canceled displacement value based upon the reference value and the displacement value; and
determining a characteristic of the tissue specimen based upon the reference-canceled displacement value.

23. The method of claim 22, wherein determining the displacement value comprises multiplying the displacement signal by a linear factor and by a non-linear factor.

24. The method of claim 23, wherein determining the displacement value does not comprise frequency filtering the displacement signal before multiplying the displacement signal by the linear factor.

25. The method of claim 22, wherein determining the reference value comprises multiplying the reference signal by a linear factor.

26. The method of claim 22, wherein determining the characteristic comprises multiplying the reference-canceled displacement value by a linear factor.

27. The method of claim 26, wherein the linear factor is a correlation between displacement of the first post and the characteristic of the tissue specimen.

28. The method of claim 22,
wherein determining the displacement value comprises multiplying the displacement signal by a first linear factor and by a non-linear factor,
wherein determining the reference value comprises multiplying the reference signal by a second linear factor, and
wherein determining the characteristic comprises multiplying the reference-canceled displacement value by a third linear factor.

29. The method of claim 22, wherein the characteristic is an absolute force.
